(19) United States
(12) Patent Application Publication
(10) Pub. No.: US 2022/0220227 A1
(43) Pub. Date: Jul. 14, 2022

(54) Chitosan and Applications Thereof

(71) Applicant: KIOMED PHARMA, Herstal (BE)

(72) Inventors: Mickaël Chausson, Huy (BE); Laurence Hermitte, Bouc Bel Air (FR); Vincent Hamers, Neuville-En-Condroz (BE); Sandrine Gautier, Liege (BE); Pierre Douette, Embourg (BE)

(21) Appl. No.: 17/614,099

(22) PCT Filed: May 20, 2020
(86) PCT No.: PCT/EP2020/064159

§ 371 Date: Nov. 24, 2021

(30) Foreign Application Priority Data
May 24, 2019 (FR) FR1905504

(51) Int. Cl. C08B 37/08; C08L 5/08; C08J 3/24; A61L 27/20; A61L 27/48; A61L 27/26

(52) U.S. Cl. CPC C08B 37/003; A61L 2430/06; C08B 37/0072; C08J 3/24; A61L 27/20; A61L 27/48; C08J 3/246; A61L 27/26; A61L 2400/06; C08L 2201/08; A61L 2430/02; C08L 2203/02; C08L 2205/025; C08J 2305/08; C08J 2405/08; C08L 5/08

(57) ABSTRACT

The present invention relates to a crosslinked carboxyalkyl chitosan forming a matrix, compositions comprising same, a method for manufacturing same, and the different applications thereof, in particular in the field of therapy, rheumatology, ophthalmology, aesthetic medicine, plastic surgery, internal surgery, dermatology, gynecology or cosmetics. The invention relates in particular to a matrix comprising at least one carboxyalkyl chitosan having glucosamine units, N-acetylglucosamine units and glucosamine units substituted with a carboxyalkyl group, the carboxyalkyl chitosan having a degree of acetylation ranging from 40% to 80%, expressed as the number of moles of N-acetyl groups relative to the number of moles of total glucosamine units, the carboxyalkyl chitosan being crosslinked by covalent bonds between the chains of carboxyalkyl chitosan.

The present invention relates to a crosslinked carboxyalkyl chitosan, forming a matrix, compositions comprising it, its manufacturing process and its various applications, in particular in the therapeutic, rheumatological, ophthalmological, aesthetic medicine, plastic surgery, open surgery, dermatological, gynecological or cosmetic field.

### STATE OF THE ART

Chitosan derivatives are known, especially in the Kiomed Pharma applications published under WO 2016/016463 and WO 2016/016464 and the corresponding patents. Also known from Kiomed Pharma are advantageous chitosan derivatives such as carboxyalkyl chitosans described in Kiomed Pharma patent applications filed as PCT/EP2018/080763 and PCT/EP2018/080767 and their family, the contents of which are incorporated into the present invention by reference.

It would be advantageous according to the present inventors to be able to adjust biomechanical behavior of the carboxyalkyl chitosan compositions, or even to increase the life time or the effect of treatment by the presence of carboxyalkyl chitosan. However, it is not obvious to the person skilled in the art to provide such compositions with improved biomechanical properties, in particular when it is desired to prepare a hydrogel. Among biopolymer compositions and especially hydrogels of the state of the art, one of the technical problems of biopolymer-based compositions, known to those skilled in the art, lies in the fact that some compositions are not in the form of a cohesive hydrogel, that is, the hydrogel spontaneously disintegrates into distinct parts in the presence of an aqueous medium, thus forming particles, fragments. This is also known as a fragmented gel or hydrogel.

It is recognized that such non-cohesive hydrogels present risks of long-term inflammatory nodule formation or granulomatous reaction when the product is implanted in human or animal tissues, considered undesirable for many medical applications (Bergerey-Galley, Aesth Surf J 24, 33, 2004). It is therefore important in terms of the health safety of the subject or patient to be able to avoid formation of distinct fragments and to obtain compositions in the form of cohesive hydrogels.

Moreover, it is desired to avoid such aggregates in some cases, for several reasons, to improve the aesthetic (visual and/or to the touch) appearance of the tissues being filled with such a composition, properly biointegrated into the tissue or tissues allowing a homogeneous filling.

Thus, for many applications, a cohesive hydrogel is preferred, which remains in one block, for example, when an aqueous medium is added thereto. This is also referred to as a “homogeneous” hydrogel. Furthermore, for most applications, a hydrogel is preferred that is referred to as a “smooth” hydrogel due to its visual appearance with no or few lumps.

In addition to cohesion, a composition according to the invention, and in particular a hydrogel, should be suitable for use in humans or animals, especially in terms of safety, immunocompatibility, bioresorbability, biomechanical properties and life time or activity time. However, not all compositions in the state of the art satisfactorily exhibit such properties and would therefore not be in accordance with the present invention.

Various methods for implementing carboxylalkyl chitosans into hydrogel form are known. Especially, Rufato et al. (Intechopen 81811, 2018), Upadhyaya et al. (J Controlled Release 2014), and Fonseca-Santos et al. (Mater Sci Engineering C 77, 1349, 2017) have identified several chitosan-based hydrogels, including carboxylalkyl chitosans, for medical or pharmaceutical uses. However, none of these hydrogels are what the present inventors are looking for, as they do not meet expectations, in particular simultaneously, in terms of cohesion, safety, immunocompatibility, biomechanical properties, bioresorbability and/or life time or activity time. None of the carboxyalkyl chitosans used to prepare known hydrogels according to the state of the art exhibit good immunocompatibility according to the inventors, apart from Kiomed Pharma compositions according to the aforesaid applications PCT/EP2018/080763 and PCT/EP2018/080767. Not just any chitosan can be used to form hydrogels acceptable for use in humans or animals.

Chitosan-based hydrogels known to date are prepared by combining chitosan or one of the derivatives with other polymers, for example, alginate, isopropylacrylamide, polyurethane, polyacrylonitrile, gelatin, Polyethylene glycol (PEG), polyvinyl alcohol (PVA). However these polymers are either non-bioresorbable or immunoreactive, which does not meet the purposes of the invention.

For example, Huang et al. (RCS Adv 2016 D01:10.1039/C5RA26160K) prepared a glycol chitosan and hyaluronan hydrogel, however such a glycol chitosan is not acceptable in humans because it is immunoreactive. Song et al (Sci Rep 6, 37600, 2016) prepared a hydrogel based on carboxymethyl chitosan and oxidized hyaluronan, via a Schiff base reaction between the amine groups of carboxymethyl chitosan and the hyaluronan aldehydes. However, in the experience of the present inventors, the carboxymethyl chitosan used does not have the molecular structure required to satisfy the purposes of the present invention. In particular, the described hydrogel resorbs very rapidly, according to the in vitro and in vivo tests set forth. Such a hydrogel therefore needs to be improved especially with respect to its life time in order to be used for a wide range of indications.

Furthermore, previous products are often not very versatile to meet the needs of different indications, especially different therapeutic indications. There is therefore a need to provide a product that is sufficiently versatile in terms of properties, especially biomechanical properties, to easily adapt it to different applications.

For example, in regenerative medicine or surgery, it is generally sought to repair an altered tissue or fluid and/or to prevent tissue alterations, to fill a tissue, or even to separate tissues to avoid adhesions. The origin of the tissue alteration can be natural aging, an external aggression (trauma, UV radiation, surgery . . . ), a pathology, for example inflammatory, autoimmune pathology, etc. But most tissue alterations involve oxidating stress, sometimes called oxidative stress, characterized by a high content of free radical species capable of damaging the tissue or cells. Reducing the amount of free radical species allows the tissue to prevent/delay its aging and to reduce harmful consequences thereof. There are several ways to reduce the amount of free radical species in a tissue, for example by administering antioxidant substances, such as vitamins C, B, E, and/or ubiquinone. Alternatively, a composition capable of scavenging free radicals can be used, thereby reducing their content and propagation in the tissue.

Chitosan and some of its derivatives exhibit the ability to scavenge oxidative free radical species, as described for many formulations for biomedical use, as listed in the review by Ngo et al. (Adv Food Nutrition Res 73, 15, 2014). For example, carboxymethyl chitosans with different structure and molecular weight have been studied for their ability to scavenge different types of free radicals using in vitro measurement methods, as described especially by Ujang et al. (The Development, Characterization and Application of Water Soluble Chitosan; in Biotechnology of Biopolymers, InTech, 2011. ISBN: 978-953-307-179-4).

However, it is difficult to provide compositions for applying the beneficial effects of chitosan, especially its ability to scavenge free radicals, in the form of treatments allowing both reduction of the impact of oxidative stress on tissues and better adjustment of the biomechanical behavior of the product, or even increase the life time or the effect of the treatment by the presence of this polymer of exogenous origin.

Thus, the state of the art does not obviously allow those skilled in the art to provide a satisfactory composition to overcome problems set out in the present invention.

### Purposes of the Invention

One purpose of the invention is to solve the technical problem of providing a chitosan derivative or a composition comprising it, suitable for use in a human or animal, in particular in the therapeutic, surgical and cosmetic fields.

One purpose of the invention is to solve the technical problem consisting in providing a chitosan derivative or a composition comprising it, for applying the beneficial effects of chitosan, especially its ability to scavenge free radicals, in the form of a treatment making it possible both to reduce impact of oxidative stress on the tissues and better adjust biomechanical behavior and increase the life time or the effect of the treatment through the presence of this polymer of exogenous origin.

In particular, one purpose of the invention is to solve the technical problem of providing a composition, especially in the form of a bioresorbable hydrogel, adapted to be used in contact with a human or animal tissue, acceptable in terms of biomechanical properties, in situ life time or activity time, good health safety (in particular absence of immunological reaction and/or foreign body reaction in the short and long term) and having beneficial effects, in particular in the context of regenerative medicine or anti-aging medicine, for example in the therapeutic, rheumatological, orthopedic, gynecological, ophthalmological, aesthetic medicine, plastic surgery, open surgery, dermatological, or cosmetic fields.

One purpose of the invention is to solve the technical problem of providing a composition with good biomechanical properties, and in particular biomechanical properties that are adjustable according to its indication.

One purpose of the invention is to solve the technical problem of providing a product based on a chitosan derivative allowing to prepare a range of products with variable biomechanical properties, adapted to each intended indication.

One purpose of the invention is to solve the technical problem of providing a composition providing, preferably simultaneously, cohesion, safety (including immunocompatibility), biomechanical properties, bioresorbability sufficient for administration in a human or animal, and preferably with an appropriate life time or activity time.

One purpose of the invention is to solve the technical problems set out in the present invention by providing in particular a chitosan derivative or a composition comprising it with a grade acceptable for human or animal in the intended indication.

### DETAILED DESCRIPTION OF THE INVENTION

To solve the technical problems set out in the invention, the inventors sought to develop a chitosan having both good antioxidizing properties and good mechanical properties for the intended applications in humans or animals (referred to as biomechanical properties).

The inventors have the knowledge from their own experience of the advantages of a substituted chitosan, especially a carboxyalkyl chitosan. In particular, Kiomed Pharma has filed patent applications under PCT/EP2018/080763 and PCT/EP2018/080767. They have sought to apply this teaching to solve the technical problems set out in the invention.

It has been noticed by the present inventors that a carboxyalkyl chitosan hydrogel formed by ionic (that is, non-covalent) crosslinking does not retain its biomechanical properties long enough after implantation for some intended applications; especially, this technology does not allow for broad modulation of life time or activity time. Furthermore, carboxyalkyl chitosan hydrogels formed by enzymatically catalyzed crosslinking have a risk of enzyme immunoreactivity due to its proteinaceous nature and complicates final purification of the resulting crosslinked product.

Patent application CN 107325306 (Imeik Technology Development) describes the preparation of gels based on carboxymethyl chitosan of crustacean origin by crosslinking with BDDE in several successive crosslinking steps (multi-crosslinking). However, this method does not provide a hydrogel according to the criteria of the invention in particular because the hydrogel obtained is not cohesive since it is formed by particles of crosslinked chitosan derivatives which are dispersed in a solution of carboxymethyl chitosan, the whole being crosslinked again to form a gel. The crosslinking operation is repeated several times (“multi-crosslinking”). Such a product is likely to form granulomas and thus to affect immunocompatibility negatively after contact with the human or animal body, which is precisely what the invention seeks to avoid. The invention further advantageously allows for greater versatility of indications, especially when a cohesive (that is, remaining in one block and not fragmenting, for example, upon contact with water) and/or “smooth” appearance hydrogel is desired. According to CN 107325306, the carboxymethyl chitosan used has a low DA (degree of deacetylation of 60-99%, preferably 80-95%, that is a degree of acetylation (DA) much lower than 40% in practice). Carboxymethyl chitosan hydrogels with a low degree of acetylation are also described by Czechowska-Biskup et al. (D01: 10.15259.PCACD.21.03). However, these hydrogels are not cohesive and do not meet the purposes of the present invention.

The inventors have discovered that a crosslinked carboxyalkyl chitosan matrix according to the invention or a composition, especially a hydrogel, comprising it makes it possible to solve at least one, and preferably all, of the technical problems set out in the invention.

Thus, the invention relates to a matrix comprising at least one carboxyalkyl chitosan having glucosamine units, N-acetylglucosamine units and glucosamine units substituted with a carboxyalkyl group, said carboxyalkyl chitosan having a degree of acetylation ranging from 40% to 80%, expressed as the number of moles of N-acetyl groups relative to the number of moles of total glucosamine units, said carboxyalkyl chitosan being crosslinked by covalent bonds between the carboxyalkyl chitosan chains.

Indeed, it has been discovered that a crosslinked carboxyalkyl chitosan with a DA of less than 40% did not make it possible to obtain a hydrogel with the desired cohesion, in that it fragmented into separate fragments during moisturization, which is undesirable for many applications.

According to the present invention, a cohesive hydrogel is understood to be a hydrogel that retains its cohesion according to the following cohesion test called the ‘water test’, by adapting methods conventionally used to characterize hydrogels for intradermal use, for example the one described by Micheels et al (J Clin Aesth Dermatol 10, 29, 2017 and J Drugs Dermatol 15, 1092, 2016):

A 1 g mass of the hydrogel to be tested is placed in the center of a 5 cm diameter glass Petri dish. A volume of 1 mL of distilled water is added to the periphery of the dish. The Petri dish is gently oscillated until the water covers the hydrogel and then returned to the horizontal position. It is observed whether the hydrogel remains integral immediately after contact of the matrix with the water, and preferably after a contact of 15 to 25 seconds, and preferably after a contact of at least 30 seconds, that is, forms a single piece when it is cohesive, or whether it spontaneously separates into distinct parts, or forms particles visible to the naked eye when it is non-cohesive.

Furthermore, it has been advantageously discovered that matrices according to the invention are capable of scavenging free radical species. The preservation of this chitosan property was far from obvious to those skilled in the art. While it is known that the molecular structure (DS) and molecular weight of carboxyalkyl chitosan influence its ability to scavenge free radicals, contradictory results have been published. Therefore, it was not clear that a crosslinked carboxyalkyl chitosan exhibits free radical scavenging ability.

Furthermore, the hydrogels according to the invention exhibit such antioxidant activity, while having appropriate cohesion, biomechanical profile, longevity and safety.

Furthermore, it was not clear that a crosslinked carboxyalkyl chitosan formulated as a hydrogel would be cohesive, preferably smooth, that is, without distinct, visible or perceptible fragments to the touch, and have appropriate safety, in particular, immunocompatibility, biomechanical profile and longevity. The invention makes it possible to provide such a matrix or composition, especially in the form of a hydrogel. For a crosslinked matrix to be immunocompatible, that is, non-immunoreactive and which does not substantially activate an immune reaction, it should at least be prepared from a non-immunoreactive polymer or polymers. To verify that a polymer is non-immunoreactive, specific and standardized tests are used, for example the human whole blood test (in vitro) and subcutaneous injection into the air bag in mice.

It may be accepted that a hydrogel formed by a matrix according to the invention is not completely smooth and has, for example, visible or perceptible lumps to the touch, provided that it is cohesive according to the aforesaid water test.

A matrix according to the present invention may be characterized by the starting carboxyalkyl chitosan, which is crosslinked to form a matrix according to the invention.

According to a first aspect, a carboxyalkyl chitosan of fungal origin is used having carboxyalkyl-substituted glucosamine units, N-acetyl glucosamine units and glucosamine units, said carboxyalkyl chitosan preferably having a degree of substitution with a carboxyalkyl group of greater than 20%, expressed as the number of moles of the substituent relative to the number of moles of total units.

This is also referred to as a chitosan derivative or substituted chitosan.

Carboxyalkyl chitosan is prepared by substitution of chitosan. Typically, a carboxyalkyl chitosan is prepared according to Kiomed Pharma patent applications filed under PCT/EP2018/080763 and its family (especially FR 17 61314 and EP 18799772.1) and PCT/EP2018/080767 and its family (especially FR 17 61323 and EP 18799773.9), which are incorporated herein by reference in particular to illustrate preparation of a carboxyalkyl chitosan.

Chitosan is, for example, referenced under CAS number 9012-76-4.

The chitosan used for the invention is advantageously of fungal origin, and preferably derived from the mycelium of a fungus of the Ascomycete type, and in particular Aspergillus piger, and/or a Basidiomycete fungus, and in particular Lentinula edodes (shiitake) and/or Agaricus bisporus (white mushroom). Preferably, the chitosan is derived from Agaricus bisporus. The chitosan is preferably highly pure, that is containing few impurities from its fungal origin or from the manufacturing process, and of a microbiological grade compatible with its use as an implant or pharmaceutical composition. One method for preparing chitosan is that described in patents WO 03/068824 (EP 1483299; U.S. Pat. No. 7,556,946).

In general, chitin is suspended in aqueous medium in the presence of sodium hydroxide, then the medium is heated at high temperature for a variable time depending on the desired molecular weight. The chitosan is then purified by solubilization in acid medium and precipitated in alkaline medium, washed and dried.

Preferably, the chitosan is of sufficiently pure grade for pharmaceutical use.

The chitosan is advantageously purified and then preferably dried. After purification, the process of the invention may include a step of drying the carboxyalkyl chitosan and then optionally grinding it to a powder. The carboxyalkyl chitosan may be dried, for example, by evaporating water, for example, by a spray-drying (atomization), fluidized bed process, or by heat drying under vacuum or atmospheric pressure, or by lyophilization.

The carboxyalkyl chitosan may be solubilized in an aqueous solution, and for example in pharmaceutical grade water acceptable for injection or implantation into a body, and in particular a human body.

Such a carboxyalkyl chitosan is then crosslinked to prepare a matrix according to the invention.

The DA and DS of the crosslinked carboxyalkyl chitosan can be expressed as a function of DA and DS of the uncrosslinked carboxyalkyl chitosan because DA and DS do not vary substantially upon crosslinking. However, if the crosslinking agent provides N-acetyl or carboxyalkyl groups, these groups which are foreign to the starting uncrosslinked carboxyalkyl chitosan are not taken into account in DA and DS of the crosslinked carboxyalkyl chitosan. The values of DA and DS are known to the person skilled in the art, as explained below. The DA and DS are therefore referred to both before and after crosslinking.

The degree of acetylation (DA) of chitosan is determined as for example described in patent applications WO2017009335 and WO2017009346 by potentiometric titration. Alternatively, the DA can be measured by other methods known for chitosan, such as liquid phase proton NMR, solid phase carbon-13 NMR, infrared spectrometry.

Advantageously, the carboxyalkyl chitosan has a degree of acetylation of between 40 and 80%, expressed as the number of moles of N-acetyl glucosamine units relative to the number of moles of total units. The degree of acetylation is expressed as the number of N-acetyl groups (of the D-glucosamine units) relative to the number of total glucosamine units present in the chitosan (N-acetyl-D-glucosamine, substituted N-acetyl-D-glucosamine, D-glucosamine and substituted D-glucosamine).

Advantageously, the carboxyalkyl chitosan has a degree of acetylation of between 40 and 80%, expressed as the number of N-acetyl groups relative to the number of total glucosamine units.

According to one alternative, the degree of acetylation ranges from 40 to 50%. According to one alternative, the degree of acetylation ranges from 50 to 60%.

In one embodiment, the degree of acetylation ranges from 60 to 75%.

The degree of acetylation of the carboxyalkyl chitosan can be determined by solid phase carbon-13 NMR or by solid phase Carbon-13 NMR or by liquid phase proton NMR. The carboxyalkyl chitosan advantageously has a controlled degree of acetylation. By the terms “chitosan having a controlled degree of acetylation”, it is meant a product whose degree of acetylation, that is the proportion of N-acetyl-glucosamine units, can be adjusted in a controlled manner, especially by an acetylation reaction.

Preferably, the carboxyalkyl chitosan is reacetylated.

According to one alternative, the process for preparing the carboxyalkyl chitosan according to the invention comprises preparing a chitosan of fungal origin, reacetylating the chitosan and carboxyalkylating the chitosan reacetylated. Thus, the invention relates to a reacetylated carboxyalkyl chitosan. In particular, the invention relates to an anionic carboxyalkyl chitosan.

According to one embodiment, chitosan can thus be dissolved in an aqueous, preferably slightly acidified, medium (pH 6, for example). Acetic anhydride can be added to the chitosan solution in one or more steps. A basic agent such as soda and/or urea is then added. Then an alkylating agent such as, for example, sodium monochloroacetate (that is, the sodium salt of chloroacetic acid) or chloroacetic acid is added. Then the substituted chitosan is purified, recovered and dried.

According to one alternative, the process for preparing carboxyalkyl chitosan according to the invention comprises preparing a chitosan, carboxyalkylating the chitosan and then reacetylating the chitosan carboxyalkylated. Advantageously, such a method allows precise control of the degree of acetylation of the final carboxyalkyl chitosan, and in particular to obtain a high degree of acetylation, for example above 40%. Thus, the invention relates to a chitosan reacetylated and then carboxyalkylated or a carboxyalkyl chitosan reacetylated.

According to one alternative, the process for preparing the carboxyalkyl chitosan according to the invention comprises preparing a chitin of fungal origin, carboxyalkylating the chitin, and optionally reacetylating the chitin carboxyalkylated to obtain the carboxyalkyl chitosan according to the invention.

According to one alternative, the process for preparing the chitosan carboxyalkylated according to the invention comprises preparing a chitin of fungal origin, deacetylating the chitin, carboxyalkylating the chitin, and optionally reacetylating the chitin carboxyalkylated to obtain the carboxyalkyl chitosan according to the invention.

According to one alternative, the carboxyalkyl chitosan has an average molecular weight of less than 400,000.

According to one alternative, the average molecular weight is between 20,000 and 60,000.

According to another alternative, the average molecular weight is between 60,000 and 120,000.

According to another alternative, the average molecular weight is between 100,000 and 400,000.

According to another alternative, the average molecular weight is between 120,000 and 400,000.

According to another alternative, the average molecular weight is between 180,000 and 400,000.

Preferably here, the average molecular weight is the viscosity average molecular weight (Mv), calculated from the inherent viscosity. This expression is customary to those skilled in the art. The inherent viscosity (n) is measured by capillary viscometry, with a capillary viscometer of the Ubbelohde type, according to the method in monograph 2.2.9 of the European Pharmacopoeia. The flow time of the solution is measured through an adapted capillary tube (Lauda, for example Ubbelohde 510 01 capillary tube with a diameter of 0.53 mm) using an automatic I-Visc viscometer (Lauda). To calculate the average viscometric mass of the carboxyalkyl chitosan, the Mark-Houwink equation (η=K*Mva^{α}) is then applied, where:
Mv is the viscosity-average molecular weight of carboxyalkyl chitosan,
η is the intrinsic viscosity of carboxyalkyl chitosan,
the constants K and α have a value of 0.0686 and 0.7638, respectively, as previously determined for (unsubstituted) chitosan by steric exclusion chromatography with a MALLS detector.

Thus, the inherent viscosity of carboxyalkyl chitosan can usually be expressed:

It is possible to hydrolyze the chitosan to decrease its molecular weight.

Typically, in uncrosslinked carboxyalkyl chitosan, the glucosamine units are D-glucosamine units (D-glucosamine units, N-acetyl-D-glucosamine units, and at least one of the D-glucosamine units and the N-acetyl-D-glucosamine units being substituted).

According to one alternative, a substituted chitosan has a substitution of the D-glucosamine units only.

According to another alternative, a substituted chitosan has substitution of the D-glucosamine and N-acetyl-D-glucosamine units simultaneously, and wherein the carboxyalkyl group is covalently bonded, according to one alternative to the amine groups of the chitosan only, or according to another alternative to the amine and hydroxyl groups of the chitosan simultaneously.

The substitution is generally only partial, not all units are necessarily substituted.

According to one embodiment, the degree of substitution of the D-glucosamine units expressed as the number of moles of D-glucosamine units relative to the number of moles of total units (D-glucosamine and N-acetyl-D-glucosamine units, substituted or not) of the substituted chitosan, ranges from 30% to 250%.

According to one embodiment, said carboxyalkyl chitosan has a degree of substitution with a carboxyalkyl group greater than 20%, for example greater than 50%, for example less than 200%, expressed as the number of moles of the substituent relative to the number of moles of total units.

According to one embodiment, the degree of substitution with a carboxyalkyl group is greater than 50%, expressed as mole number of the substituent relative to the mole number of total units.

According to one embodiment, the degree of substitution of the D-glucosamine units expressed as the number of moles of D-glucosamine units with respect to the number of moles of total units (D-glucosamine and N-acetyl-D-glucosamine units, substituted or unsubstituted) of the substituted chitosan, ranges from 50% to 200%, and still preferably is higher than 70%.

According to one embodiment, the degree of substitution with a carboxyalkyl group is less than 80%, expressed as the number of moles of the substituent relative to the number of moles of total units.

Typically, the substitution is achieved by covalent bonding.

According to one alternative, the carboxyalkyl chitosan is an N,O-carboxyalkyl chitosan. The proportion of units substituted with a carboxyalkyl group in the O-position (either O3 or O6 of the glucosamine and/or N-acetyl-glucosamine units) and/or in the N-position (of the glucosamine units) varies. The degree of substitution can therefore be greater than 100%.

Advantageously, the degree of substitution (DS) and the degree of acetylation (DA) of the carboxyalkyl chitosan are measured by solid phase carbon-13 NMR, using a Bruker Spectrometer (Avance III HD 400 MHz), equipped with a PH MAS VTN 400SB BL4 N-P/H probe. For example, the spectrum is recorded at room temperature, a relaxation time between 1 and 8 seconds, a number of scans between 64 and 512. The areas of the signals of the carbons are determined after deconvolution. The carbons considered are: “CH3 acetyl” (methyl carbon of the acetyl group of the N-acetyl-glucosamine units, substituted or not), “Cx” (carbon in position×of the glucosamine and N-acetyl-glucosamine units, x ranging from 1 to 6) and “C═O” (carbonyl carbon of the carboxyalkyl substituent and C═O carbonyl carbon of the acetyl group of the N-acetyl-glucosamine units, substituted or not). To determine DS of a given carboxyalkyl chitosan, the carbon 13 NMR spectrum of the precursor chitosan of this carboxyalkyl chitosan should also be recorded. From the spectrum of the precursor chitosan, the “CSU ratio”, that is, the ratio of the signal area of the “CH3 acetyl” group (methyl carbon of the acetyl group of the N-acetyl-glucosamine units) to the signal area of “C═O” (carbonyl carbon of the acetyl group of the N-acetyl-D-glucosamine units) is calculated. DA of the carboxyalkyl chitosan is calculated according to Formula 1, and DS according to Formula 2, where I represents the signal area of the carbon under consideration.

$$\begin{matrix}[ Math 1 ] & \\ DA = \frac{{I}_{{CH}_{3} acetyl}}{\sum {I}_{Cx} / 6} & Formula 1 \\ [ Math 2 ] & \\ DS = \frac{{I}_{C = O - {I}_{{CH}_{3} acetyl}} / CsU ratio}{\sum {I}_{Cx} / 6} & Formula 2\end{matrix}$$

DA and DS can be determined using other methods known for carboxyalkyl chitosans, for example, by proton NMR in aqueous medium using a magnetic resonance spectrometer, for example, according to the method described by Liu et al. (Carb Polym 137, 600, 2016), for example, with prior hydrolysis of the carboxyalkyl chitosan adding to it a concentrated solution of deuterated hydrochloric acid before analysis.

If another NMR method is more advantageous to reliably estimate DA and/or DS, such a method is suitable for use. The above methods should be adapted by the skilled person relating to sample preparation and the signals to be integrated, especially with respect to the resolution, robustness, and proton position of the signals to be used for calculating the degree of substitution.

The degree of carboxyalkylation of chitosan can advantageously range from 20 to 250%, preferably from 50 to 200%, and for example from 70 to 170%, expressed as the number of moles of carboxyalkyl relative to the number of moles of total units.

According to one alternative, the degree of carboxyalkylation of the chitosan can advantageously range from 40 to 130%, and for example from 70 to 130%, expressed as the number of moles of carboxyalkyl with respect to the number of moles of total units.

The degree of substitution of chitosan is typically correlated to the mass ratio of reactants to chitosan upon starting the reaction. Examples of carboxyalkylating agents include acid chlorides (or salts thereof, for example, sodium monochloroacetate), such as those bearing one or more carboxymethyl, carboxyethyl, carboxypropyl, carboxybutyl groups etc.

According to one alternative, the present invention relates to a carboxyalkyl chitosan wherein the alkyl portion of the carboxyalkyl is linear or branched, C1-C5.

According to one embodiment, the present invention relates to a carboxymethyl chitosan.

According to this alternative, the substituted chitosan is an N-carboxyalkyl chitosan.

According to this embodiment, the substituted chitosan is an O-carboxyalkylated chitosan.

According to this alternative, the substituted chitosan is an N-carboxyalkylated and O-carboxyalkylated chitosan.

According to a second aspect, the present invention relates to a chitosan derivative having glucosamine units, N-acetyl-glucosamine units and glucosamine units substituted with a carboxyalkyl group, said carboxyalkyl chitosan having a zeta potential, measured at pH 7.5, less than or equal to −10 mV, and preferably less than or equal to −15 mV. Especially, such a chitosan derivative makes it possible to limit the immune response of a subject to which the chitosan derivative or a composition comprising it has been administered, typically by instillation, injection or implantation.

Advantageously, the zeta potential, measured at pH 7.5, is less than or equal to −18 mV.

Advantageously, the carboxyalkyl chitosan has a zeta potential, measured at pH 7.5, less than or equal to −22 mV, and preferably less than or equal to −24 mV.

According to one specific alternative, the substituted chitosan preferably has an average molecular weight of 150,000 to 220,000 and a degree of substitution ranging from 50 to 200%, the molecular weight preferably being expressed before substitution.

According to another specific alternative, the substituted chitosan has an average molecular weight of 120,000 to 150,000 and a degree of substitution ranging from 70 to 200%, the molecular weight preferably being expressed before substitution.

According to one specific alternative, the substituted chitosan preferably has an average molecular weight of 220,000 to 300,000 and a degree of substitution ranging from 70 to 200%, the molecular weight preferably being expressed before substitution.

According to another specific alternative, the substituted chitosan has an average molecular weight of 220,000 to 300,000 and a degree of substitution ranging from 50 to 200%, the molecular weight preferably being expressed before substitution.

According to another specific alternative, the substituted chitosan has an average molecular weight of 300,000 to 500,000 and a degree of substitution ranging from 50 to 200%, the molecular weight preferably being expressed before substitution.

According to another specific alternative, the substituted chitosan has an average molecular weight of 300,000 to 500,000 and a degree of substitution ranging from 70 to 200%, the molecular weight preferably being expressed before substitution.

According to one specific alternative, the substituted chitosan has preferably an average molecular weight of 120,000 to 150,000 and a degree of substitution ranging from 20 to 50%, the molecular weight preferably being expressed before substitution.

According to another specific alternative, the substituted chitosan has an average molecular weight of 220,000 to 300,000 and a degree of substitution ranging from 20 to 50%, the molecular weight preferably being expressed before substitution.

According to another specific alternative, the substituted chitosan has an average molecular weight of 300,000 to 500,000 and a degree of substitution ranging from 20 to 50%, the molecular weight preferably being expressed before substitution.

According to a specific alternative, the substituted chitosan has a degree of substitution ranging from 20 to 80%, and preferably from 40 to 60%, and a degree of acetylation of 40 to 80%, and preferably from 50 to 75%.

According to a specific alternative, the substituted chitosan has a degree of substitution ranging from 50 to 200%, and preferably from 70 to 200%, and a degree of acetylation from 40 to 80%, and preferably from 50 to 75%.

According to another specific alternative, the substituted chitosan has a degree of substitution ranging from 90 to 200%, and preferably from 90 to 150%, and a degree of acetylation of 40 to 80%, the molecular weight preferably being expressed before substitution.

According to a specific alternative, the substituted chitosan has a degree of substitution ranging from 90 to 200%, and preferably from 90 to 150%, and a degree of acetylation from 40 to 60%, and preferably from 50 to 60%.

According to one specific alternative, the substituted chitosan has a degree of substitution ranging from 90 to 200%, and preferably from 90 to 150%, and a degree of acetylation of 50 to 75%.

According to one specific alternative, the substituted chitosan preferably has an average molecular weight of 220,000 to 300,000, a degree of substitution ranging from 90 to 200%, and preferably 90 to 150%, and a degree of acetylation of 50 to 75%, the molecular weight preferably being expressed before substitution.

By substituting chitosan, it has been possible to prepare a solution of a carboxyalkyl chitosan soluble in an aqueous solution the pH of which varies within a wide range, whereas unsubstituted chitosan is only soluble at pH below 5.5 to 6.5. Thus, carboxyalkyl chitosan exhibits an ability to be solubilized at different pHs by virtue of the presence of carboxyalkyl groups that modify its solubility profile, and in particular at physiological pH or at the pH of physiological fluids modified by a pathology, for example an inflammatory pathology.

By “water soluble” it is meant that the carboxyalkyl chitosan does not show any visible cloudiness to the naked eye when placed in aqueous solution. More specifically, the solubility, that is, the absence of cloudiness, of a solution of carboxyalkyl chitosan at a concentration of, for example, 1% (m/m) in water or a buffer, for example, a phosphate buffer, can be confirmed by an optical density of less than 0.5, and preferably less than 0.2, measured by UV-visible spectrometry at a wavelength of 500 nm with reference to a reference cell only comprising the aqueous solvent used for the sample measured, but in the absence of substituted chitosan. Another method consists in a visual inspection according to monograph 2.9.20 of the European Pharmacopoeia. When the chitosan is not sufficiently substituted, the composition is not soluble in a satisfactory pH range, for example ranging from pH 5.5 to pH 8.5, at room temperature.

According to one embodiment, the carboxyalkyl chitosan is sterile.

It is especially understood by “crosslinked by covalent bonds between the carboxyalkyl chitosan chains” that the chitosan main chain (also called chitosan backbone) is covalently bonded to one or more main chitosan chains. Advantageously, a three-dimensional network of chitosan molecules is thus obtained. The invention is not limited to a particular covalent crosslinking method, but a method using a chemical molecule as crosslinking agent, also known as a crosslinking agent, is preferred.

According to the invention, carboxyalkyl chitosan is crosslinked.

According to one alternative, the crosslinks are formed by a crosslinking agent forming said covalent bonds.

Thus, several chitosan chains can be crosslinked, for example by reaction with one or more crosslinking agents, such as, for example, selected from crosslinking agents used for crosslinking polysaccharides, such as 1,4-butanediol diglycidyl ether, 1-bromo-3,4-epoxybutane, 1-bromo-4,5-epoxypentane, 1-chloro-2,3-epithio-propane, 1-bromo-2,3-epithiopropane, 1-bromo-3,4-epithio-butane, 1-bromo-4,5-epithiopentane 2,3-dibromopropanol, 2,4-dibromobutanol, 2,5-dibromopentanol, 2,3-dibromopro-panethiol-1,2,4-dibromobutanethiol, and 2,5-dibromopentane-thiol epichlorohydrin, 2,3-dibromopropanol-1,1-chloro-2,3-epithiopropane, dimethylaminopropylcarbodiimide, gallic acid, epigallocatechin gallate, curcumin, tannic acid, genipin, or even diisocyanate compounds such as hexamethylene diisocyanate or toluene diisocyanate, or even divinyl sulfone.

Genipin is a naturally occurring crosslinking agent used to crosslink polysaccharides, especially carboxymethyl chitosan (Yang et al. Acta Pharmacol Sin 31, 1625, 2020). Genipin colors the hydrogel a dark blue to black color, which may be an advantage in some indications.

Preferably, the crosslinking agent is a polyepoxide type, for example difunctional agent. Preferably, 1,4-butanediol diglycidyl ether (BDDE) or ethylene glycol diglycidyl ether (EGDE) is used as the crosslinking agent, as they are already used for the preparation of biomaterials applied in humans, especially hyaluronan hydrogels for intradermal, intra-articular or intra-ocular administration. According to one alternative, the crosslinking agent is divinyl sulfone.

Advantageously, the composition of the invention may also comprise a biopolymer other than crosslinked carboxyalkyl chitosan. According to an advantageous alternative, the biopolymer is a polysaccharide, oxidized or not, crosslinked by covalent bonds or not, for example a glycosaminoglycan, and in particular a hyaluronan such as for example hyaluronic acid or sodium hyaluronate.

The advantage of combining or crosslinking a crosslinked carboxyalkyl chitosan with some other polymers is to add their biological and physicochemical properties, or even to create synergies.

According to one alternative, the matrix according to the invention comprises a crosslinked carboxyalkyl chitosan and a hyaluronan, a chondroitin sulfate and/or a carboxymethyl cellulose. To date, there is no hydrogel of crosslinked carboxyalkyl chitosan (as defined for the invention) combined with a hyaluronan. It is one of the objects of the invention to combine these two polymers in order to be able to combine, for example, recognized moisturizing properties of hyaluronan with the protective properties against oxidative stress of chitosan.

According to one alternative, the matrix comprises at least one hyaluronan.

Advantageously, the matrices according to the invention comprise crosslinked carboxymethyl chitosan alone or a crosslinked carboxymethyl chitosan combined with a hyaluronan, crosslinked or not. This makes it possible to adapt the desired properties.

Said matrix comprises at least one carboxymethyl chitosan and a hyaluronan.

According to one alternative, the hyaluronan has an average molecular weight of less than 5 million and preferably greater than 1 million, preferably greater than 2 million, as determined by capillary viscometry. The molecular weight of the hyaluronan is sometimes expressed via its density, as they are correlated via a linear relationship. The hyaluronan may have a density of up to 4.25 m³/kg, and for example be designated as of low density (for example, about 1 to 2 m³/kg) or high density (for example, about 2 to 4 m³/kg).

According to one alternative, the hyaluronan is obtained by fermentation, for example with Streptococcus. According to another alternative, it is produced by extraction from rooster peaks.

According to one alternative, the matrix comprises at least one hyaluronan crosslinked by covalent bonds.

Thus, the crosslinked hyaluronan comprises covalent bonds between different hyaluronan chains.

Different types of hyaluronan can be crosslinked with each other, such as hyaluronans with different molecular weights or different hyaluronan salts.

The present invention also relates to a process for preparing the crosslinked carboxyalkyl chitosan.

According to one alternative, the process for preparing a matrix according to the invention, comprises:

contacting the carboxyalkyl chitosan, with at least one crosslinking agent, contacting preferably being carried out in an alkaline aqueous phase;

crosslinking the carboxyalkyl chitosan with the crosslinking agent; and

obtaining a matrix comprising the carboxyalkyl chitosan crosslinked.

According to one alternative, the carboxyalkyl chitosan is crosslinked in an alkaline aqueous phase, for example in the presence of a sodium hydroxide (NaOH) solution.

Advantageously, the concentration of carboxyalkyl chitosan initially present in the aqueous phase is in the range of from 1 to 30%, and preferably from 5 to 20% (w/v) by weight of carboxyalkyl chitosan relative to the volume of alkaline aqueous phase.

Advantageously, the mass ratio between the crosslinking agent and the polymer(s) is from 0.1% to 30%, expressed by weight of the crosslinking agent relative to the weight of the polymer(s).

Preferably, the mass ratio between the crosslinking agent and the polymer(s) is from 0.5% to 20%, in particular when BDDE is used, expressed as weight of the crosslinking agent relative to the weight of the polymer(s).

Typically, the reaction is carried out with heating, for example at a temperature of 25 to 60° C., and for example 50° C., for example over a period of 30 minutes to 48 hours, for example 1 hour to 5 hours. In general, crosslinking is stopped by neutralizing and diluting, for example by adding an acid, and for example by adding acetic acid or a hydrochloric acid.

Advantageously, the reaction residues are removed by dialysis using a phosphate salt buffer.

A hydrogel comprising a matrix according to the invention is thus obtained.

On the other hand, carboxyalkyl chitosan is an exogenous molecule that is more resistant to degradation than hyaluronan after implantation/injection/instillation into a body.

Thus, the invention relates to a matrix comprising a three-dimensional network based on these two polymers having different molecular weights. Thus, advantageously, a range of biomechanical properties, in situ product duration and treatment duration are provided, while retaining the free radical scavenging power of carboxyalkyl chitosan.

The invention relates to a matrix comprising at least one hyaluronan co-crosslinked by covalent bonds with carboxyalkyl chitosan.

According to one alternative, the process for preparing a matrix comprising a carboxyalkyl chitosan, preferably as defined according to the invention, co-crosslinked with another biopolymer, and preferably a hyaluronan, said process comprising:

contacting a mixture of carboxyalkyl chitosan, and the other biopolymer, and preferably hyaluronan, with at least one crosslinking agent, the contacting preferably being carried out in the alkaline phase;

crosslinking the carboxyalkyl chitosan and the other biopolymer, and preferably a hyaluronan, with the crosslinking agent;

obtaining a co-crosslinked matrix of carboxyalkyl chitosan and the other biopolymer, and preferably a hyaluronan.

According to one alternative, a matrix according to the invention is sterile.

It is advantageous to provide a hydrogel from a matrix according to the invention.

Thus the invention relates to a hydrogel, and advantageously forms a cohesive hydrogel.

The present invention thus relates to crosslinked carboxyalkyl chitosan hydrogels in which the carboxyalkyl chitosan has a high degree of acetylation (DA) (greater than 40%), and preferably also has a high degree of substitution (DS) (greater than 20%, preferably greater than 50% and typically less than 200%).

The invention relates to a composition comprising at least one matrix defined according to the invention.

According to a preferred alternative, a matrix according to the invention is formulated in an aqueous medium to form a composition in the form of a hydrogel.

Advantageously, the concentration of polymer (carboxyalkyl chitosan with or without another biopolymer, such as a hyaluronan, for example) is less than 10%, for example less than or equal to 5%, by mass relative to the total mass of the composition, and in particular of the hydrogel (m/m).

According to one alternative, the concentration of polymer (carboxyalkyl chitosan with or without other biopolymer, such as hyaluronan) is less than 4%, for example less than or equal to 3%, by mass with respect to the total mass of the composition, and in particular of the hydrogel (m/m).

The mass ratio (m/m) [carboxyalkyl chitosan/hyaluronan] is, for example, from 5 to 95%, for example from 10 to 90%, and still for example from 30 to 70%. The mass ratio (m/m) [hyaluronan/carboxyalkyl chitosan] is for example from 5 to 95%, for example 10 to 90%, and further for example 30 to 70%. According to one alternative, the mass ratio (m/m) [carboxyalkyl chitosan/hyaluronan] is 1:1 (that is 50% chitosan and 50% hyaluronan).

The aqueous medium can be water, an aqueous solution, whose pH and osmolality are for example adjusted using an acid/base buffer system with the addition of salts and/or optionally polyols (sorbitol, mannitol, glycerol).

According to one alternative, the matrix according to the invention is formulated in a hydrolipidic medium allowing the formation of a single or multiple, direct or reverse emulsion.

According to one embodiment, the composition of the matrix has an osmolality of 100 to 700 mosm/kg, preferably 120 to 500 mosm/kg.

Advantageously, the osmolality of the composition of the matrix is from 250 to 400 mosm/kg, and preferably from 270 to 330 mosm/kg.

According to one alternative, the composition of the matrix has an osmolality suitable for a joint.

According to one alternative, the composition of the matrix has an osmolality compatible with an ocular or intraocular surface.

According to one alternative, the composition of the matrix has an osmolality compatible with a dermis or mucosa.

According to one alternative, it is preferable that the osmolality of the composition of the matrix is between 100 and 400, and more specifically between 120 and 380 mosm/kg.

According to one alternative, a composition according to the invention is sterile.

Advantageously, the composition according to the invention is contained in an injection, implantation, or instillation device such as a syringe or vial.

Advantageously, the injection device, such as a syringe for example, can then undergo steam sterilization. This device, such as a syringe, can then be packaged, preferably in an aseptic or sterile way. It may also be a bag, a flapula, or a vial for instilling the composition according to the invention, aseptically filled after sterilizing the formulation, or directly sterilized after filling.

According to one alternative, a composition according to the invention, and in particular a hydrogel according to the invention, is sterilized by filtration and/or by steam sterilization, before filling an injection, implantation or instillation device, such as a syringe or a vial.

The person skilled in the art knows techniques for sterilizing a hydrogel to obtain a desired sterile hydrogel. They have several types of equipment for heat or steam sterilization, and can use several types of cycles that remove the microbial load.

The present invention more particularly relates to an injectable composition comprising a matrix, preferably in the form of a hydrogel, according to the invention.

The invention also relates to a pharmaceutical composition comprising at least one matrix, preferably in the form of a hydrogel, according to the invention.

According to one alternative, the composition according to the invention is used as an injectable, implantable or instillable pharmaceutical composition, or injectable or implantable or instillable medical device.

The invention further covers a composition according to the invention in a dry form, especially in a lyophilized form. The lyophilized product can especially be (re)dispersed, and preferably solubilized, before use.

The present invention more particularly relates to a composition according to the invention for use in therapeutic treatment, for example comprising injecting by subcutaneous, intradermal, intraocular, or intra-articular, intra-mucosal, intra-muscular route said composition, for example for the repair, regeneration or filling of at least one body tissue/liquid requiring repair or filling.

It is advantageous to use a chitosan having a sufficient degree of purity for the application contemplated.

It is advantageous to use a hyaluronan with a degree of purity sufficient for the application contemplated.

The biomechanical properties sought by the composition according to the invention may vary in nature amplitude depending on the indication, for example depending on the tissue in which the hydrogel is to be integrated, the mechanism of action or the effect intended to ensure benefit for the patient, and duration of the effect.

Advantageously, the properties of the composition according to the invention and in particular of a hydrogel according to the invention are adapted to the indication. In order to adapt these properties, the final concentration of polymers (carboxyalkyl chitosan and/or other biopolymers such as a hyaluronan), and/or the rate of crosslinking, especially via the crosslinking agent/polymers mass ratio, and/or the nature and/or quantity of the ions, and/or the initial molecular weight of the polymer(s), are varied, for example.

In particular, the invention relates to a highly elastic hydrogel, especially when a lasting increase in volume at the cutaneous, subcutaneous or periosteal level (for projection or remodeling) has to be ensured, or a viscoelastic gel, especially to allow both shock absorption and a lubricating effect at the articular level. The invention relates to a lubricating hydrogel, especially when it is necessary to reduce friction between two biological surfaces, for example two cartilage surfaces in a joint, or the ocular surface and the eyelids in an eye. A composition of the invention may have a variable elasticity level, adjusted according to the indication, and may be characterized by measuring the modulus of elasticity by rheometry.

Preferably, the matrix has an antioxidant capacity by scavenging free radicals, especially a normalized antioxidant capacity greater than 0.30, preferably greater than 0.50, and even more preferably greater than 0.80, and for example greater than 0.90.

The present invention relates to an injectable composition characterized in that it comprises at least one matrix defined according to the invention.

The present invention relates to a pharmaceutical composition characterized in that it comprises at least one matrix defined according to the invention.

According to one alternative, the composition according to the invention is used as an injectable, implantable or instillable, or topically administrable pharmaceutical composition, or an injectable or implantable or instillable, or topically administrable medical device, for example for use in a therapeutic treatment method, for example comprising topically instillating or administrating or injecting said composition by subcutaneous, intradermal, mucosal, ocular, intraocular, or intra-articular, intraosseous route, for example for the repair or filling of at least one body tissue in need of repair or filling.

According to one alternative, the composition according to the invention is used in a method for the treatment, repair or filling of at least one body fluid or tissue in need of repair or filling, and for example whose body tissue is selected from tissues belonging to the vocal cords, muscles, ligaments, tendons, mucous membranes, sexual organs, bones, joints, eyes, dermis, or any combination thereof, and in particular dermis, cartilage, synovial membrane, a skin wound or even the ocular surface.

The present invention relates to a composition according to the invention for use in a method for treating osteoarthritis, or repairing a cartilage defect, for example by injection into a biological fluid, for example synovial fluid, or after mixing with a biological fluid, for example blood, and implantation into cartilage. By biological fluid, it is meant a fluid of body origin which may or may not have undergone a treatment modifying its composition.

The present invention relates to a medical device, for example a medical implant, characterized in that it comprises or consists of a composition as defined according to the invention.

The present invention relates in particular to a composition according to the invention for use in therapeutic, surgical or cosmetic treatment, including in particular treatment in rheumatology, ophthalmology, gynecology, aesthetic medicine, plastic surgery, open surgery, orthopedic surgery, gynecology, for the prevention of post-surgical tissue adhesions, and in dermatology.

The present invention also relates to a composition according to the invention for use in the therapeutic treatment of dry eye syndrome, corneal injury or ocular or joint inflammation.

The present invention further relates to the application of a composition according to the invention by instillation on the ocular surface to prevent or combat corneal injury, or dry eye syndrome, in particular for the purpose of lubricating or regenerating the ocular surface.

Thus, the invention also relates to an eye drop composition comprising a carboxyalkyl chitosan defined according to the present invention.

According to one alternative, the subject is afflicted by an inflammatory pathology (for example osteoarthritis, arthritis, dry eye syndrome).

The present invention more particularly relates to a composition according to the invention for the treatment of arthrosis, arthritis, or repair of a cartilage defect, for example by injection into the synovial cavity or by implantation at the cartilage defect.

The present invention more particularly relates to a medical device, for example a medical implant, characterized in that it comprises or consists of a composition according to the invention.

According to one preferred alternative, the invention thus relates to a medical device comprising a chamber containing a composition according to the invention in a dry form, especially in a lyophilized form, and optionally one or more other chambers containing one or more active products, additives or excipients.

The composition according to the present invention may also comprise one or more active agents for a desired indication, and/or one or more additives or excipients for modulating the properties of the composition according to the invention.

The present invention also relates to a composition according to the invention for use in a therapeutic treatment method.

The present invention also relates to a composition according to the invention for use in a method for treating arthrosis, or repairing a cartilage defect, for example by injection into the synovial sac or after mixing with blood and implantation into the cartilage/bone.

The present invention also relates to a composition according to the invention for use in an aesthetic treatment or care method by dermal filling (“dermal filling”) or lip filling. This involves especially, for example, injecting a composition according to the invention subcutaneously, intradermally, intramucosally or intramuscularly.

The present invention also relates to a composition according to the invention for use in a method for superficial treatment of the skin by multiple intradermal injections, or of other tissues, according to conventional mesotherapy methods well known to those skilled in the art. Such compositions can typically be used in dermatology, as treatments for aesthetic purposes. The purpose of such a method is, for example, to plump up the skin to make it lose a wrinkled appearance (treatment of wrinkles and/or fine lines). Such a treatment can be intended for a subject wishing to give a rejuvenated appearance to his/her skin.

The present invention also relates to a composition according to the invention for use in a treatment method in which the composition is a viscosupplementation agent. Here, for example, the composition of the invention is injected intra-articularly, especially to limit friction of the cartilage surfaces of the joint.

The present invention also relates to a composition according to the invention for use as a cell vector, of one or more cell types, and/or one or more active agents. These may be active agents from a pharmaceutical or biological point of view. The composition of the invention may indeed be compatible with the presence of cells, preferably living cells. Examples of living cells of interest include: chondrocytes (articular cartilage), fibrochondrocytes (meniscus), ligament fibroblasts (ligament), skin fibroblasts (skin), tenocytes (tendons), myofibroblasts (muscle), mesenchymal stem cells, red blood cells (blood) and keratinocytes (skin). The composition of the invention may also be targeted as a therapeutic vector for targeted and/or controlled release delivery of at least one therapeutic agent.

According to one alternative, blood, or plasma, or platelet lysate, or platelet-rich plasma, or any biological fluid is added with the composition of the invention for example for increasing the performance of the product.

According to one alternative, the composition according to the invention is formulated in a solid form (for example, a film or a porous foam), which swells/moisturizes once implanted (for example, tear plug, dressing).

According to one alternative, the composition is formulated in a form of a nebulizable composition (spray).

The present invention also relates to a composition according to the invention for use in a method for the treatment or cosmetic care of one or more tissues or organs affected by excessive temperature, as in the case of a burn.

The present invention also relates to a composition according to the invention for use in a method for treating cartilage repair (for example, by implantation on a cartilage defect with a view to promoting its regeneration).

The present invention also relates to a composition according to the invention for use in a method for the preventive treatment of tissue adhesions after surgery: the product is applied to the tissues at the end of surgery, for example gynaecological, abdominal, visceral, orthopaedic, etc.

The invention relates to a physiological composition, administered topically, by injection or by implantation, for contacting with one or more living tissues subjected to oxidative stress, for example:
intra-articular injection for the treatment of osteoarthritis (via synovial fluid supplementation, cartilage lubrication, shock absorption at the joint level, regeneration of the synovial membrane); intra-articular implantation to promote repair of cartilage defects;
intraosseous implantation to promote bone repair (osteoinduction/osteoconduction);
subcutaneous and/or intradermal injection for filling or regenerating the skin or hair follicles, to increase volume in cases of lipoatrophy;
ocular instillation to relieve ocular surface symptoms or prevent alterations, for example for the treatment of dry eye and corneal lesions, and the administration of active principles;
intraocular injection, for example for optimizing effectiveness of glaucoma surgery or vitreous-supplementation, as an adjuvant to cataract surgery, for regeneration of anterior or posterior ocular tissues, and intraocular administration of active principles;
administration on internal tissues and organs (films) to prevent post-surgical adhesions;
administration on wounds, cracks, tears, cavities . . . of tissues and organs such as skin, bones, cartilage, cornea, tendons, meniscus . . . with a view to promoting their repair or regeneration;
injection into the vulval mucosa for the treatment of vulvodynia.

The present invention also relates to a composition according to the invention forming an artificial synovial fluid.

The composition according to the present invention makes it possible to mimic a healthy synovial fluid or to improve a healthy or defective synovial fluid by seeking, for example, to improve its lubricating ability to reduce friction in the joint, and/or its shock-absorbing properties (identifiable by the elastic modulus G′), while at the same time being easily injectable, for example to fill a syringe, or to be injected into the human or animal body. As an indication, the elastic modulus G′ of healthy synovial fluid is between 40 and 100 Pa, and its loss modulus G″ is between 1 and 10 Pa.

Advantageously, for intra-articular injection, a composition according to the invention is easily injectable through a fine needle, for example a 21 Gauge diameter needle, at room temperature. By “easy” injection, it is preferably meant that the force to be exerted on such a syringe is less than 50 Newton (at a speed of 10 mm/min) to flow a composition according to the invention through a 21 Gauge needle, preferably a force of less than 20 Newton.

Advantageously, for intradermal injection, a composition according to the invention is easily injectable through a fine needle, for example a 25 Gauge or smaller diameter needle, at room temperature. By “easy” injection, it is preferably meant that the force to be exerted on such a syringe to eject into the air is less than 30 Newton (at a speed of 10 mm/min) to flow a composition according to the invention through a 27 Gauge needle, preferably a force of less than 20 Newton.

The present invention also relates to a composition as artificial tears comprising a carboxyalkyl chitosan according to the invention.

In general, the ranges of osmolality and pH values of the composition are adapted, and in general close to the osmolality and pH values of the tissues in contact with the composition according to the invention.

Advantageously, the composition according to the present invention is sterile. Very advantageously, the composition according to the present invention is sterilized by temperature rise, preferably under autoclave.

According to one embodiment, the matrix has a lubricating ability with a low coefficient of friction (COF), for example less than 20, and for example less than 10, according to the test of the examples of the invention.

According to one alternative, the compositions of the invention are transparent or translucent.

By “translucent”, it is meant that an object can be distinguished when its composition is placed between the observers eye and the object. By “transparent”, it is understood that alphanumeric characters can be distinguished when the composition is placed between the observer's eye and the observed characters. In general this evaluation is carried out with a thickness of composition of approximately 1 cm. The method of the monograph 2.9.20 of the European Pharmacopoeia for the visual inspection can also be adopted. The optical density of the composition can also be measured, for example by UV-visible spectrometry at 500 nm and make sure that the optical density is less than 0.5, preferably 0.2 relative to a reference solvent.

According to one alternative, the compositions of the invention are not or only slightly opalescent.

By “opalescent”, it is meant that the solution causes diffraction of light visible to the naked eye, for example by visual inspection according to a method such as monograph 2.9.20 of the European Pharmacopoeia and by comparison with reference solutions of different opalescence levels of the European Pharmacopoeia. According to one alternative, the composition of the invention is colorless, that is in particular, an observer with the naked eye does not assign a specific color to the composition. According to one alternative, the opalescence is below the maximum tolerated for the application contemplated.

The invention relates in particular to preferably sterile articles or packaging, comprising one or more instillation or injection devices pre-filled with a composition according to the invention, in particular in the form of a hydrogel. These are typically devices for instilling the product in the form of drops or pre-filled syringes.

A composition of the invention can advantageously be stored, preferably in an article or packaging suitable for its indication, and preferably for several months.

Advantageously, the composition of the invention can be sterilized. Thus, the invention relates to a sterilized crosslinked carboxyalkyl chitosan. The crosslinked carboxyalkyl chitosan is thus sterile, especially for applications requiring it.

According to one alternative, the composition of the invention is steam sterilized, according to a method known to the person skilled in the art and/or recommended by the European Pharmacopoeia.

According to another alternative, the composition may be sterilized by filtration using filters intended for this purpose, for example filters with a porosity less than or equal to 0.2 μm.

Advantageously, according to a preferred embodiment, the loss in intrinsic viscosity of the crosslinked carboxyalkyl chitosan upon steam sterilization is less than 40%.

The present invention also covers a method for therapeutic treatment comprising injecting a composition according to the invention.

The present invention also covers the use of a composition according to the invention for the preparation of a pharmaceutical composition, in particular for a therapeutic treatment, for example as more specifically defined by the invention.

The present invention also covers a method for aesthetic, in other words non-therapeutic, treatment comprising injecting a composition according to the invention. This is, for example, filling of wrinkles or filling of one or more damaged visible tissue zones, for example as a result of an accident or a surgical procedure, for aesthetic purposes.

A tissue is a group of similar cells of the same origin, gathered in a functional unit, that is, they all contribute to the same function. Among the tissues mention can be made of: dermal tissue (for example epithelial tissue), connective tissue, muscular tissue, and nervous tissue.

By “composition according to the invention” or equivalent terms, it is meant a composition defined as in the present invention, including according to any of the alternatives, particular or specific embodiments, independently or in any combination thereof, including according to the preferred characteristics.

Further purposes, characteristics and advantages of the invention will become clearer to the person skilled in the art upon reading the explanatory description, which refers to examples that are given for illustrative purposes only and are in no way intended to limit the scope of the invention.

The examples are an integral part of the present invention and any characteristic that appears to be novel in relation to any prior art from the description as a whole, including the examples, is an integral part of the invention in its function and generality.

Thus, each example has a general scope.

On the other hand, in the examples, all percentages are given in mass unless otherwise indicated, and temperature is expressed in degrees Celsius unless otherwise indicated, and pressure is atmospheric pressure unless otherwise indicated.

### EXAMPLE

Method for Measuring the Zeta Potential

The formulation to be analyzed is diluted in a phosphate buffer to obtain a final concentration of polymer of 0.05%, then gently stirred until homogenization. The solution is then separated into different fractions, and the pH of each fraction is adjusted to the desired value, between pH 4 and 8, either by adding 0.1N sodium hydroxide or 0.1N hydrochloric acid. The zeta potential of each fraction is measured using a “Nano-Z” apparatus (Zeta-Sizer range, Malvern Instruments).

Method for Measuring the Solubility Range of Chitosan Polymers

The solubility range is established by preparing a solution of the polymer to be tested at a concentration of 1% and a pH of 9, by fractionating it into several fractions whose pH is adjusted to different pH values over a range from 9 to 1. For each fraction, the polymer is checked for solubility, that is, it does not form cloudiness, according to the visual inspection method of monograph 2.9.20 of the European Pharmacopoeia. The pH range over which the polymer is soluble or insoluble is noted.

Biomechanical Profile by Rheometry

The biomechanical profile of the sample is characterized using a DHR-2 Hydrid Rheometer (TA Instrument) equipped with a 20 mm plane geometry spaced 700 μm from the Peltier, at a temperature of 37° C., a frequency of 3.98 rad/s and a deformation amplitude ranging from 0.1 to 10%. Each measurement is performed in triplicate, and then the average value of the moduli of elasticity (G′), viscosity (G″), and tan 8 (G″/G′) of the three measurements is calculated.

Lubricating Ability

The lubricating ability is characterized by the coefficient of friction (COF) between two surfaces. The measurement of the coefficient of friction is carried out according to the following method, the parameters of which are chosen according to the intended product and the indication.

Method for Viscosupplements

Two discs based on a polyacrylate biomaterial used for the manufacture of hydrophobic intraocular lenses (as described in patent EP 1830898) with a diameter of 16.15 mm are previously moisturized by immersion in water at 60° C. for about 2 hours, and then fixed on the upper and lower geometries of a DHR-2 rheometer (TA Instruments). A volume of about 100 μL of the sample to be tested is placed on the lower disc, then the upper geometry is lowered until contact between both discs, up to an imposed normal force of 5 Newton. Coefficient of friction measurements are performed at 25° C. for a duration of 150 seconds, at constant normal force (5N), oscillation frequency of 1.256 rad/s, and deformation angle of approximately 0.05 radian, according to a protocol adapted from the protocol described by Waller et al. (in: J 47 Rheumatol 39, 7, 1473, 2012). The option “adherence to the zero starting point of the oscillatory motion” is activated. At each measurement point, the torque value is recorded, and then the coefficient of friction (COF) is calculated according to the formula: COF=torque/(⅓×disc diameter×normal force). For each formulation, the measurement is replicated 5 times. The value of the coefficient of friction is reported by extrapolation 5 of the intercept at the start of each COF versus time curve (COF₀).

Method for Artificial Tears

Two discs based on a polyacrylate biomaterial used for the manufacture of hydrophobic intraocular lenses (as described in patent EP 1830898) with a diameter of 16.15 mm are previously moisturized by immersion in water at 60° C. for about 2 hours, and then fixed on the upper and lower geometries of a DHR-2 rheometer (TA Instruments). A volume of about 100 μL of the sample to be tested is placed on the lower disc, and then the upper geometry is lowered until contact between both discs, up to an imposed normal force of 5 Newton. Coefficient of friction measurements are performed at 25° C. for a duration of 150 seconds, at constant normal force (5N), oscillation frequency of 1.256 rad/s, and deformation angle of approximately 0.05 radian, according to a protocol adapted from the protocol described by Waller et al.

(in: J 47 Rheumatol 39, 7, 1473, 2012). The option “adherence to the zero starting point of the oscillatory motion” is activated. At each measurement point, the torque value is recorded, and then the coefficient of friction (COF) is calculated according to the formula: COF=torque/(⅓×disc diameter×normal force). For each formulation, the measurement is replicated 5 times. The value of the coefficient of friction is reported by extrapolation of the intercept at the start of each COF versus time curve (COF₀).

Ejection Force Via a Needle

The measurement is performed using a MultiTest 2.5-i compression tester (Mecmesin) equipped with a 100N compression cell. A suitable needle is adapted to the syringe containing the sample. The syringe is positioned on the tester, the plunger of the syringe is pressed at a constant speed (for example 10 or 80 mm/min), and the force required for ejection is measured. The maximum force tolerated by the equipment is about 70 Newton.

In Vitro Antioxidant Capacity (ABTS Test)

To measure the antioxidant activity of carboxyalkyl chitosan formulations and compare it with commercially available products, the in vitro ‘ABTS’ test is applied. This test consists in determining the capacity of a substance to trap the cation radical of 2,2′-azino-bis(3-ethylbenzothiazoline-6-sulfonic acid) (ABTS 1), a chromophore whose maximum absorption is located at the wavelength 734 nm in its cation radical form. The protocol is adapted from the method described by Valyova et al (Int J Applied Res Nat Prod, 5, 19, 2012) and performed with a Nunclon 96 type polystyrene microplate (Thermo Fisher Scientific) and an Infinite M200 microplate reader (Tecan Life Sciences) for absorbance measurement.

Each test series is performed in 4 steps.

1) 1 g of 2,2′-azino-bis(3-ethylbenzothiazoline-6-sulfonic acid) diammonium salt (ABTS) is diluted in a homogeneous solution of K₂S₂O₈ (2.45 mM in MilliQ water) to obtain a concentration of 7 mM ABTS. This mixture is protected from light and stirred at room temperature for 24 hours, the time required to generate a defined amount of ABTS 1 radical cations. The ABTS 1 working solution is finally obtained by taking 600 μL of the latter mixture and diluting this amount in MilliQ water to a concentration of 415 μM.

2) A calibration curve of the free radical scavenging capacity is established by comparison with Trolox, a reference antioxidant molecule (6-hydroxy-2,5,7,8-tetramethylchroman-2-carboxylic acid). Solutions of Trolox with concentrations of 30, 60, 90, 120, 150, 180 and 210 μM are obtained by diluting in MilliQ water a stock solution of 15 mg of Trolox in 5 mL of 100% methanol. Absorbance measurements are performed at wavelength 734 nm, 1 hour after mixing 50 μL of ABTS 1 working solution and 50 μL of each Trolox solution. The relationship between absorbance and Trolox concentration in the linearity zone is read out. The minimum absorbance value in the linearity zone corresponds to the detection limit.

3) The products to be tested are either characterized as such at their initial concentration, or diluted in MilliQ water (to be defined according to the product to be tested so that the absorbance of the mixture with the ABTS 1 solution is higher than the detection limit). 50 μL of the working solution and 50 μL of the test product solution are mixed. The absorbance is measured at wavelength 734 nm after 1 hour incubation at room temperature. If the absorbance value is within the detection range of the apparatus, it is retained and the Trolox equivalent is calculated via the calibration curve, noted TEAC for “trolox equivalent antioxidant capacity”.

4) A positive control is used to express the antioxidant capacity in a normalized way from one series to another, ascorbic acid (vitamin C) in solution at the concentration 0.02 mg/mL (20 μg/mL). First, the TEAC of ascorbic acid solutions from 0.005 to 0.05 mg/mL is measured. It is verified that the absorbance of the 0.02 mg/mL ascorbic acid solution is in the linearity zone. Finally, the normalized antioxidant capacity of the tested product is expressed by the ratio TEAC (product)/TEAC (ascorbic acid at 0.02 mg/mL).

### Example 1

A carboxymethyl chitosan is produced via the carboxymethylation and acetylation reactions according to the method below, using the reaction parameters in Table 1a given by way of example. It is moreover possible to modulate molecular structure of the carboxymethyl chitosan using other reaction parameters.

Step 1: Carboxymethylation of Chitosan.

30 g of chitosan from Agaricus bisporus origin are dispersed in 600 mL of isopropanol, 41 mL of water and 163 mL of 50% sodium hydroxide (m/v). 135 g of the alkylating agent monochloroacetic acid (MCA) are dissolved in 135 mL of isopropanol, and added to the chitosan suspension. The reaction is continued at 35° C. for 23 hours. The polymer is recovered by precipitation in ethanol, then purified by cycles of solubilization in water and precipitation in ethanol. The carboxymethyl chitosan (reference CC4, Table 1b) is collected after drying in a ventilated oven.

Step 2: Acetylation of Carboxymethyl Chitosan.

A 21 g mass of CC4 is dispensed into 570 mL of water, and the pH of the solution is adjusted to pH>7. A volume of 10 mL of acetic anhydride is added, and the solution is stirred at 25° C. for 30 minutes. The pH of the solution is adjusted to pH>7, then 10 mL of acid anhydride is added. After homogenization (about 30 minutes of stirring at room temperature), the pH is adjusted to about pH 7.5. The polymer is recovered by precipitation in ethanol, then purified by solubilization cycles in water and precipitation. Carboxymethyl chitosan (reference CC3, Table 1 b) is collected after drying in a ventilated oven.

The carboxymethyl chitosans used to prepare matrices of Examples 2-11 are described in Table 1 b. CC1 through CC6 are carboxymethyl chitosans derived from fungal chitosan, and prepared according to the above method.

CC7 is a commercial crustacean-derived carboxymethyl chitosan provided by the Kraeber Company (product code 5313009900, Ellerbek, Germany).

TABLE 1a
Table 1a - Parameters of the carboxymethylation
and acetylation reactions

| Step 1: | |
|---|---|
| Carboxymethylation | Reference: CC4 |
| Alkylating agent | Monochloroacetic acid (MCA) |
| NaOH/chitosan | 5.44% (m/m) |
| Alkylating agent/chitosan | 4.50% (m/m) |
| Isopropanol/alkylating agent | 100% |
| Temperature - duration | 35° C. - 23 hours |
| Step 2: Acetylation | |
| Acetic anhydride/CC | Reference: CC3 |
| First addition | 0.5 (v/m) |
| Second addition | 0.5 (v/m) |
| Temperature - duration | 25° C. - 1 hour |

TABLE 1b
Table 1b - Carboxymethyl chitosans (CC)

| | | DA ^{a} | DS ^{a} | DN ^{b} | Inherent |
|---|---|---|---|---|---|
| Reference | Origin | (mol %) | (mol %) | (mol %) | viscosity ^{c} |
| CC1 | Agaricus | 58 | 82 | 20 | 1096 |
| CC2 | bisporus | 56 | 81 | 20 | 994 |
| CC3 | | 55 | 87 | 20 | 912 |
| CC4 | | 14 | 85-90 | 41 | 652 |
| CC5 | | 57 | 88 | 23 | 960 |
| CC6 | | 47 | 85 | 24 | 1064 |
| CC7 | Crustacean | <10% ^{d} | 79 | 95 | 332 |
| (Kraeber) | | | | | |

^{a} measured by solid phase carbon-13 NMR (formula 2);
^{b} measured by potentiometric titration;
^{c} measured by capillary viscometry;
^{d} acetyl group signal is not detectable by carbon-13 NMR (low DA).

### Example 2—Matrices of Carboxymethyl Chitosan

Synthetic tests have been performed to provide matrices of carboxymethyl chitosan by covalent crosslinking using the crosslinking agent 1,4-butanediol diglycidyl ether (CAS 245-79-8, BDDE). Several carboxymethyl chitosans of Agaricus bisporus origin produced by Kiomed Pharma according to the method of Example 1 are used. Their characteristics are found in Table 1. BDDE (96%, specific gravity 1.049) is supplied by Alfa Aesar (ThermoFischer, Kandel, Germany).

### Example 2a

A crosslinked matrix is prepared from carboxymethyl chitosan CC3 after the reaction parameters have been adjusted (Table 2a, reference M1-A). CC3 has a degree of acetylation of 55% and a degree of carboxymethylation of 87%, measured by carbon-13 NMR (formula 2). After dialysis, the hydrogel formed by the matrix is transferred into 3 mL glass syringes which are steam sterilized via a short cycle in a SYSTEC-DX-65 autoclave (condition “A2”). The final polymer concentration of the resulting sterilized hydrogel (M1-A) is determined by mass balance. The cohesive character of the hydrogel is analyzed by the water test and its viscoelasticity level (on a scale from 1 to 4) is determined by rheometry. The higher the score, the more viscoelastic the matrix forming the hydrogel. It is concluded that after adapting the reaction parameters, it is possible to obtain a matrix of BDDE-crosslinked carboxyalkyl chitosan forming a cohesive hydrogel according to the water test. The hydrogel has an elasticity score of 1. It is injectable through an intradermal needle (27 G 13 mm).

These same reaction parameters were then applied with two carboxymethyl chitosans of different molecular structure and whose degree of acetylation is lower than 40% (Table 1b): CC4 of fungal origin (Kiomed Pharma) and CC7 of crustacean origin (Kraeber).

TABLE 2a
Table 2a - Matrices of crosslinked carboxyalkyl chitosan

| Reference | M1-A | M1-B | M1-C | M1-D |
|---|---|---|---|---|
| Polymer - reference | CC3 | CC4 | CC4 | CC7 |
| (mass), initial | (7 g) | (7 g) | (7 g) | (7 g) |
| concentration | 11% | 11% | 11% | 11% |
| DA | >40% | <40% | <40% | <40% |
| Origin | Agaricus | Agaricus | Agaricus | Crustacean |
| | bisporus | bisporus | bisporus | |
| Aqueous phase - | Phosphate buffer | | | |
| Composition | NaOH 0.25M | | | |
| Aqueous phase - | 63.4 mL | | | |
| Volume | | | | |
| Crosslinking agent - | BDDE | BDDE | BDDE | BDDE |
| Molecule (volume) | 1.2 mL | 1.2 mL | 0.6 mL | 1.2 mL |
| Agent/polymer | 18% m/m | 18% m/m | 9% m/m | 18% m/m |
| (g/100g) | | | | |
| Temperature, duration | 50° C., 2 hrs | | | |
| of the reaction | | | | |
| Neutralization | Hydrochloric acid, phosphate buffer | | | |
| Purification | Dialysis against phosphate buffer | | | |
| Sterilization | A2* | | | |
| Final concentration | 24 | 26 | 25 | 22 |
| of polymer (mg/mL) | | | | |
| Hydrogel cohesion | OK | NOK | NOK | NOK |
| (water test) | | | | |
| pH - Osmolality | 7.2-289 | / | / | / |
| (mOsm/kg) | | | | |
| Viscoelasticity | 1 | / | / | / |
| level (scale from | | | | |
| 0 to 4) | | | | |
| Easy to inject** (27G | OK | / | / | / |
| needle, 13 mm) | | | | |

*A2: short cycle (SYSTEC DX-65 autoclave);
**the ejection force is less than 30 Newton at the ejection speed of 10 mm/min.

The matrices obtained under the same conditions as the matrix M1-A, M1-B and M1-C respectively (Table 2a), did not form a cohesive hydrogel according to the water test. In contrast, the matrix M1-A is able to form a cohesive hydrogel, thus satisfying this purpose of the present invention.

### Example 2b

An attempt is made to modulate the biomechanical properties of crosslinked carboxyalkyl chitosan-based hydrogels, in particular their viscoelasticity (measured on a scale from 0 to 4). For this, matrices are prepared from CC1, CC5 and CC6 with DA above 40% (Table 1 b), by varying the molecular weight of the carboxyalkyl chitosan (expressed as inherent viscosity) and the parameters of the crosslinking reaction. The crosslinking agent (BDDE), medium, temperature and reaction time are the same as those for the matrix M1-A, as well as the neutralization and purification conditions.

TABLE 2b
Table 2b - Matrices of crosslinked carboxyalkyl chitosan with
varying viscoelasticity

| Reference | M1-E | M1-F | M1-G | M1-H | M1-1 | M1-J | M1-K |
|---|---|---|---|---|---|---|---|
| Polymer - Reference (dry | CC1 | CC1 | CC1 | CC1 | CC5 | CC5 | CC6 |
| mass) | | | | | | | |
| Initial concentration | 11% | | | | 15% | 13% | 8% |
| BDDE/polymer (g/100g) | 18% | 13% | 21% | 26% | 9% | 9% | 18% |
| Sterilization* | A2* | A2* | A2* | A2* | A2* | A2* | A1* |
| Final concentration (mg/mL) | 23 | 21 | 24 | 23 | 22 | 25 | 24 |
| Hydrogel cohesion | OK | OK | OK | OK | OK | OK | OK |
| (water test) | | | | | | | |
| pH - Osmolality (mOsm/kg) | 7.2316 | 7.3288 | 7.2287 | 7.2287 | 7.2281 | 7.2287 | 7.4319 |
| Viscoelasticity level (scale | 2 | 1 | 2 | 3 | 3 | 1 | 1 |
| from 0 to 4) | | | | | | | |
| Easy to inject** (27G needle, | OK | OK | OK | OK | OK | OK | OK |
| 13 mm) | | | | | | | |

*A2: short cycle; A1: long cycle

It appears that it is possible to vary biomechanical properties of crosslinked carboxyalkyl chitosan-based hydrogels, especially viscoelasticity, by varying reaction parameters (in particular, initial concentration of carboxyalkyl chitosan or crosslinking agent/carboxyalkyl chitosan ratio, here BDDE/carboxymethyl chitosan) as well as the molecular weight of carboxyalkyl chitosan.

### Example 3—Matrixes of Co-Crosslinked Carboxymethyl Chitosan and Hyaluronan

Matrices are sought by crosslinking a mixture of carboxymethyl chitosan of fungal origin and DA greater than 40% (Table 1 b) and hyaluronan with BDDE (“co-crosslinking”). Hyaluronan (HA) with average viscosity molecular weight of 2.2 or 2.3 million (HA1 type) and 4.3 million (HA2 type) are used (Table 3a).

TABLE 3
Table 3 - Sodium hyaluronate (HA)

| Type | Supplier | Mw* (million) | Density* (m³/kg) |
|---|---|---|---|
| HA1 | HTL Javenech | 2.2 | 2.32 m³/kg |
| | (France) | 2.3 | 2.36 m³/kg |
| HA2 | | 4.3 | 3.68 m³/kg |

*values reported by the supplier

The agent (BDDE), medium, temperature and duration of the crosslinking reaction are the same as for the matrix M1-A of Example 2, as well as the neutralization and purification conditions. The hydrogels formed by the matrices are sterilized by autoclave as described in Example 2, according to cycle A1 or A2. Several hydrogels are described by way of illustration, as other combinations and/or parameters may also lead to cohesive hydrogels. All of these hydrogels are easily injected through an intradermal needle of size 27 Gauge and length 13 mm.

### Example 3a

It is sought to demonstrate that it is possible to co-crosslink carboxyalkyl chitosan (CC) with hyaluronan (HA) to form a cohesive hydrogel. For this, a matrix is prepared from a mixture of CC and HA in a CC/HA mass ratio of 75:25 (Table 3a). The references of the CC are in accordance with the previous examples. In addition, it is sought to modulate elasticity level from 1 to 3 (on a scale from 0 to 4), by adjusting parameters of the crosslinking reaction.

TABLE 3a
Table 3a - Matrices of co-crosslinked CC and
HA (CC/HA 75:25 mass ratio)

| Reference | M2-A | M2-B | M2-C | M2-E |
|---|---|---|---|---|
| Polymers - | CC1 | CC1 | CC5 | CC1 |
| Reference (mass) | HA1 | HA1 | HA2 | HA1 |
| Initial concentration of | (11%) | (11%) | (11%) | (11%) |
| polymers (%, m/v) | | | | |
| BDDE/polymers | 13% | 18% | 13% | 13% |
| (% g/100g) | | | | |
| Sterilization* | A2 | A2 | A1 | A1 |
| Final concentration of | 23 | 23 | 24 | 23 |
| polymers (mg/mL) | | | | |
| Hydrogel cohesion | OK | OK | OK | OK |
| (water test) | | | | |
| pH - Osmolality | 7.3-314 | 7.3-317 | 7.2-286 | 7.2-287 |
| (mOsm/kg) | | | | |
| Viscoelasticity level | 2 | 3 | 2 | 1 |
| (scale from 0 to 4) | | | | |

*conditions of Example 2

It is observed that at the same BDDE/polymer ratio (18%) and the same final polymer concentration of 23 mg/mL, hydrogel the M2-B of CC co-crosslinked with 25% HA is more elastic than the hydrogel M1-A of CC alone in Example 2. It is also concluded that it is possible to vary viscoelastic properties of the co-crosslinked carboxyalkyl chitosan and HA hydrogels by varying the molecular weight of HA, the percentage of crosslinking agent, here BDDE.

### Example 3b

It is sought to obtain cohesive hydrogels from carboxyalkyl chitosan and HA co-crosslinked in variable proportion.

TABLE 3b
Table 3b - Matrices of carboxyalkyl chitosan and
HA co-crosslinked with a variable CC/HA ratio

| Reference | M2-F | M2-G | M2-H |
|---|---|---|---|
| Polymers - | CC5 | CC5 | CC1 |
| Reference | HA1 | HA1 | HA1 |
| Initial concentration | 11% | 11% | 11% |
| CC/HA mass ratio | 25:75 | 50:50 | 95:5 |
| (m/m) | | | |
| BDDE/polymers | 13% | 13% | 13% |
| (% g/100 g) | | | |
| Hydrogel cohesion | OK | OK | OK |
| (water test) | | | |
| Final concentration of | 23 | 23 | 22 |
| polymers (mg/mL) | | | |
| pH - Osmolality (mOsm/kg) | 7.3292 | 7.3293 | 7.2299 |
| Viscoelasticity level | 3 | 2 | 1 |
| (scale from 0 to 4) | | | |

It appears that it is possible to obtain cohesive hydrogels of co-crosslinked carboxyalkyl chitosan and HA in variable proportion, and that their elasticity level depends on the carboxyalkyl chitosan/HA ratio.

### Example 4—Matrices of Crosslinked Carboxymethyl Chitosan Combined with a Hyaluronan

In this example, it is sought to evaluate the possibility of forming a cohesive hydrogel from a matrix of crosslinked carboxyalkyl chitosan combined with HA. The carboxyalkyl chitosan is first crosslinked with BDDE according to a method of Example 1, and then a solution of HA (type HA1) is added thereto. The resulting hydrogel is then sterilized by autoclaving via cycle A2. (Table 4).

TABLE 4
Table 4 - Matrix of carboxyalkyl chitosan
combined with HA

| Reference | M4-A |
|---|---|
| CC | CC5 |
| Reference - | 15% |
| Initial concentration | |
| HA | HA1 |
| BDDE/polymer (g/100 g) | 13% |
| Final concentration of polymers | 22 |
| (mg/mL) | |
| Final concentration, CC/HA ratio | 19.5/0.3 |
| (mg/mL) | |
| Sterilization | A2 |
| Hydrogel cohesion (water test) | OK |
| pH - Osmolality (mOsm/kg) | 7.2-275 |
| Viscoelasticity level (scale from 0 to 4) | 3 |
| Easy to inject (27G needle, 13 mm) | Yes |

It is easy to incorporate HA into a hydrogel based on a crosslinked carboxyalkyl chitosan matrix. The resulting hydrogel is cohesive according to the water test, and has a viscoelasticity score of 3, while being easy to inject through a 27 Gauge intradermal needle.

### Example 5—Biomechanical Properties of Hydrogels

In this example, the biomechanical properties of some representative CC hydrogels from Examples 2 through 4 are characterized by rheometry (Table 5). The hydrogels are cohesive, injectable via a 27 G needle, and have elasticity levels from 1 to 3. They are compared to those of three commercial crosslinked hyaluronan-based products intended for intradermal injection for aesthetic purposes (Table 5, reference B1 to B3): B1 is a viscous solution (tan delta>1), and B2 and B3 are cohesive gels (tan delta<1) according to the water test.

TABLE 5
Table 5 - Biomechanical profile of crosslinked CC
and commercial crosslinked HA-based products.

| | | | | | Viscoelasticity |
|---|---|---|---|---|---|
| | Cp | G′ | G″ | | level (scale |
| | (mg/mL) | (Pa) | (Pa) | tan δ | from 0 to 4) |
| M1-E | 23 | 40 | 14 | 0.3 | 2 |
| M1-F | 21 | 17 | 8 | 0.5 | 1 |
| M2-A | 23 | 37 | 14 | 0.4 | 2 |
| M2-B | 23 | 127 | 21 | 0.2 | 3 |
| M2-C | 24 | 42 | 15 | 0.4 | 2 |
| M2-E | 23 | 11 | 8 | 0.8 | 1 |
| M4-A | 22 | 106 | 34 | 0.3 | 3 |
| B1 | 20 | 11 | 13 | 1.2 | — |
| B2 | 22.5 | 48 | 31 | 0.7 | 2 |
| B3 | 25.5 | 137 | 53 | 0.4 | 3 |

It is confirmed that the crosslinked carboxyalkyl chitosan-based hydrogels according to the invention have biomechanical properties, in particular a modulus of elasticity (G′), comparable to those of commercial crosslinked HA-based products intended for intradermal injection for aesthetic medicine.

### Example 6—Ability to Scavenge ABTS°1 Free Radicals (In Vitro)

It is sought to verify that matrices of crosslinked carboxyalkyl chitosan (CC) are capable of scavenging oxidative free radicals using a standard in vitro test, known as “ABTS”, in which a free radical ABTS°1 is formed and a calibration is carried out with the antioxidant substance “Trolox”. Each test product is diluted to obtain a total concentration of polymer Cp (CC, HA, or CC and HA) of 8 mg/mL, 4 mg/mL and 1 mg/milk The result is checked to ensure that it is within the detection zone of the test, and then the ability to scavenge the free radical ABTS° 1 is expressed in Trolox equivalent. The antioxidant capacity of a 20 μg/mL ascorbic acid solution (positive control) is also measured. The antioxidant capacity of each product tested is normalized according to the formula: normalized antioxidant capacity=TEAC (product)/TEAC (ascorbic acid 20 μg/mL).

For comparison, a non-crosslinked carboxyalkyl chitosan polymer in solution (CC2), and a commercial product based on a non-crosslinked HA solution (reference B6) are tested. 4 commercial products intended for intradermal injection for aesthetic purposes are also characterized: references B1 to B3 (based on crosslinked HA alone, see Table 5 of Example 5), and B4, a hydrogel based on crosslinked HA combined with a complex of several small molecules including antioxidant molecules.

Table 6 reports the results obtained at the same total concentration of polymer (Cp) of 4 mg/mL for all products.

TABLE 6
Table 6 - Antioxidant capacity via ABTS test
(normalized to ascorbic acid at 20 μg/mL)

| | | | | |
|---|---|---|---|---|
| | | | | Normalized |
| | | Initial Cp | Cp | antioxidant |
| Reference | Composition | (mg/mL) | (mg/mL) | capacity |

Positive control

| | | | | |
|---|---|---|---|---|
| / | Ascorbic acid | / | 0.02 | 1.00 |

Solutions (without crosslinking)

| | | | | |
|---|---|---|---|---|
| S1 | CC2 | 20 mg/mL | 4 | 0.76 |
| B6 | HA | 15 mg/mL | 4 | 0.15 |

Hydrogels (with crosslinking)

| | | | | |
|---|---|---|---|---|
| M1-E | CC | 23 mg/mL | 4 | 1.02 |
| M2-A | CC/HA 75:25 | 23 mg/mL | 4 | 0.97 |

Commercial products (crosslinked HA)

| | | | | |
|---|---|---|---|---|
| B1 | HA | 20 mg/mL | 4 | 0.14 |
| B2 | HA | 22.5 mg/mL | 4 | 0.17 |
| B3 | HA | 25.5 mg/mL | 4 | 0.14 |
| B4 | HA, complex | 15 mg/mL | 4 | 0.53 |

It is observed that all CC-based compositions are able to scavenge the free radical ABTS° 1 significantly, and thus act as an antioxidant, whether it is a non-crosslinked CC solution (S1) or the crosslinked CC hydrogels (M1-E and M2-A). At the same concentration of polymer, commercial HA-only products (B6, B1, B2, and B3) do not demonstrate this ability.

Surprisingly, hydrogels M1-E (CC) and M2-A (CC/HA 75:25) demonstrate the highest antioxidant capacity of all products tested, including compared to the solution S1 of uncrosslinked CC. Both hydrogels have an antioxidant capacity similar to ascorbic acid at 20 μg/milk

Among the commercial HA-based products, only B4 is able to scavenge the radical ABTS° 1 significantly, nevertheless with a capacity 2 times lower than that of M1-E and M2-A. In fact, B4 is a crosslinked hyaluronan associated with a complex of several small molecules, including antioxidants, which are responsible for the observed effect. However, as these substances are small water-soluble molecules, it is likely that they diffuse rapidly out of the B4 hydrogel after intradermal injection, and that the hydrogel will then lose its antioxidant capacity.

### Example 7—Ability of Hydrogels to Reduce Oxidative Stress in Dermal Cell Culture In Vitro

The capacity of two hydrogels based on crosslinked CC (reference M1-E, see Example 2) and co-crosslinked CC/HA (M2-A, see Example 3) to protect human dermal cells from damage caused by ‘ROS’ (reactive oxygen species) free radicals, which are radical species encountered in skin tissue under oxidative stress, is evaluated in a standard in vitro test. It is compared to that of a non-crosslinked carboxyalkyl chitosan solution and a commercial product based on crosslinked hyaluronan intended for intradermal injection for aesthetic purposes (reference B3, see Example 5).

Human dermal fibroblasts (NHDF) at about 40% of their in vitro proliferation potential are cultured in a monolayer in DMEM (Dulbecco's Modified Eagle Medium) with 10% fetal bovine serum, penicillin and streptomycin, at 37° C. in a 5% CO2 atmosphere. The culture is transferred to DMEM without fetal bovine serum and then fractionated into wells. The product to be tested is diluted in DMEM to total concentrations of polymer of 0.6 and 0.2 mg/mL and added to wells (3 wells per product to be tested). After 72 hours of contact with the product to be tested, 2′-7′-dichloro-dihydrofluorescein diacetate probe, which fluoresces under the effect of free radicals, is added for 30 minutes. The culture in each well is then rinsed with HBSS to remove the product to be tested, the cells are returned to HBSS, and then all wells are irradiated with UVA at 12.5 J/cm² for 20 minutes to generate ROS.

An untreated, non-irradiated culture is used as a reference. An untreated and irradiated culture is used as a negative control, and an ascorbic acid-treated (504/mL) and irradiated culture is used as a positive control. At the end of UVA irradiation, the fluorescence intensity (excitation wavelength 485 nm, emission 520 nm), which is proportional to the ROS content, is measured, and then the relative ROS content to the unirradiated reference is calculated (Table 7). The decrease in the ROS content relative to the untreated and irradiated control, which characterizes the ability of the product to decrease oxidative stress, is then calculated.

TABLE 7
Table 7 - Capacity to decrease oxidative stress in a culture of
human dermal fibroblasts (treatment for 72 hours before
exposure to UVA for 20 minutes)

| | | | | Relative | |
|---|---|---|---|---|---|
| | | | | ROS | |
| | | | | content | |
| | | | | (± | |
| | | | | standard | ROS |
| | | | | deviation | content |
| | | | | mean, | decrease |
| Treatment | Composition | Concentration | UVA | N = 3) | (%) |
| Reference | / | / | No | 100 ± 9% | / |
| Negative | / | / | Yes | 338 ± 20% | 0% |
| control | | | | | |
| Positive | Ascorbic | 50 μg/mL | | 233 ± 4% | −31% |
| control | acid | | | | |
| Solution | | | | | |
| S1 | non- | 0.6 mg/mL* | | 249 ± 26% | −26% |
| | crosslinked | | | | |
| | CC2 | | | | |
| Hydrogels | | | | | |
| _M1-E | Crosslinked | 0.6 mg/mL* | | 238 ± 25% | −30% |
| | CC | | | | |
| M2-A | Crosslinked | 0.6 mg/mL* | | 265 ± 24% | −22% |
| | CC/HA | | | | |
| | 75:25 | | | | |
| B3 | Crosslinked | 0.6 mg/mL* | | 307 ± 16% | −9% |
| | HA | | | | |

*Total concentration of polymer (CC, CC/HA or HA) for cell treatment

Under the in vitro culture conditions of this test, it is concluded that CC-based compositions, whether crosslinked (M1-E) or non-crosslinked (S2), have a good capacity to decrease the ROS content, that is to decrease the oxidative stress likely to alter the cells and the dermal tissue. This capacity is at the same level as that of ascorbic acid (504/mL, vitamin C), and much higher than that of the commercial crosslinked HA product. With 75% of CC, the composition M2-A of co-crosslinked CC/HA also has a good capacity to decrease oxidative stress.

### Example 8—Carboxyalkyl Chitosan Matrix-Based Fluid Hydrogel for Ocular Administration

In this example, it is sought to obtain a crosslinked CC hydrogel whose viscosity allows it to be easily instilled in the form of a well-defined drop, while having good lubricating ability suitable for an artificial tear indication for ocular surface treatment.

For this, a cohesive crosslinked CC hydrogel is prepared by targeting a dynamic viscosity in the range of 1 to 60 mPa·s (at a shear rate of 10 s⁻¹) (M8-B, Table 8a). Its instillability is verified, and its lubricating ability between two polyacrylate surfaces is measured according to the method for artificial tears, expressed as a coefficient of friction.

The properties of this hydrogel are compared with those of two commercial products based on non-crosslinked HA intended for the ocular surface treatment (references B7 and B8, Table 8b). Their lubricating ability is measured in the same test series as M8-B.

TABLE 8
Table 8a - Properties of a crosslinked
CC-based fluid hydrogel

| Reference | M8-B |
|---|---|
| Polymer - reference, | CC1 |
| initial concentration | 11% |
| BDDE/polymer | 13% |
| (g/100 g) | |
| Sterilization | A2 cycle |
| Acceptable | Yes |
| appearance/texture? | |
| Cohesion (water test) | OK |
| Final concentration | 7 mg/mL |
| of polymer (mg/mL) | |
| Instillability | OK |
| Dynamic viscosity at | 1.1 |
| 10⁻¹ s (mPa · s) | |
| Coefficient of friction | 164 ± 44 |

TABLE 8b
Table 8b - Properties of commercial HA-based
products for the treatment of the ocular surface

| Reference | B7 | B8 |
|---|---|---|
| HA concentration | 1.8 mg/mL | 3 mg/mL |
| (mg/mL) | | |
| Cohesion according to | NOK | NOK |
| water test | | |
| Instillability | OK | OK |
| Dynamic viscosity at | 6 | 50 |
| 10⁻¹ s (mPa · S) | | |
| Coefficient of friction | 141 ± 57 | 264 ± 64 |

It is concluded that cohesive, fluid and instillable crosslinked CC hydrogels with lubricating ability comparable to commercial products for ocular surface treatment can be obtained.

### Example 9—Local Effects after Intradermal Implantation in Rabbits (Short Term)

Three CC matrix-based hydrogels are evaluated by intradermal administration in rabbits: M1-A (crosslinked CC, see Example 1), M2-A and M2-B (co-crosslinked CC/HA, see Example 2). These formulations are packaged in a 1 mL glass syringe (Hypak, BD Medical) and sterilized. Their endotoxin content measured according to the monograph EP 2.6.14—method D of the European Pharmacopoeia is satisfactory. Two commercial products based on crosslinked hyaluronan intended for intradermal injection for aesthetic purposes are also evaluated (B1 and B2, see Example 5).

A formulation volume of 200 μL is administered by intradermal injection in rabbits via a 27 Gauge diameter needle, according to a protocol fulfilling the IS010993-10 standard for evaluation of primary irritation induced by an intradermal implant. A total of twelve injections per product have been performed on six rabbits. Local effects were observed daily for all injected sites, in particular the erythema level.

Table 9 reports the average level of erythema at 7 days after injection (score on a scale from 0 to 4). It is also noted whether a papule is visible at 7 days (score on a scale from 0 to 4). Macroscopic analysis or microscopic analysis (dermal histology) of the injection sites for animals euthanized at 7 days post-injection is used to evaluate the presence of product.

TABLE 9
Table 9 - Local effects 7 days after intradermal injection
of hydrogels in rabbits (6 injection sites per product tested)

| | | Local effects - | Papule | |
|---|---|---|---|---|
| | | erythema | volume | |
| | | (mean score, | (mean score, | Presence |
| | CP | scale from | scale from | in the |
| Reference | (mg/mL) | 0 to 4) | 0 to 4) | dermis |
| M1-E | 23 | 0 | 0 | Yes |
| M2-A | 23 | 0.5 | 1.0 | Yes |
| M2-B | 23 | 1 | 1.0 | Yes |
| B1 | 20 | 0 | 0 | Yes |
| B2 | 22.5 | 0.1 | 1.3 | Yes |

Intradermal injection of hydrogels is associated with the appearance of mild local effects, characterized by erythema with a maximum score of 1 on average at 7 days, on a scale from 0 to 4. This corresponds to a mild erythema level, comparable to that observed for the two commercial products. In addition, the presence of the products in the dermis has been demonstrated when the animals were euthanized and histological analyses were performed at day 7.

### Example 10—Hydrogels for Joint Viscosupplementation

In this example, the viscoelastic properties and lubricating ability of two hydrogels based on crosslinked CC (M1-E) and co-crosslinked CC/HA (M2-B) have been evaluated, and compared to that of two commercial products based on crosslinked HA intended for the treatment of osteoarthritis by joint viscosupplementation (B9 and B10, see composition in Table 10). The lubricating character of hydrogels is determined by their ability to reduce the coefficient of friction between two polyacrylate polymer discs mounted to a rheometer, according to the method for viscosupplements.

TABLE 10
Table 10 - Biomechanical profile (rheometry) and coefficient
of friction (COF, average of 3 syringes per product, 5
measurements per syringe) of hydrogels

| | Cp composition | | | | |
|---|---|---|---|---|---|
| Reference | (mg/mL)** | G′ (Pa) | G″ (Pa) | tan 8 | COF |
| M1-E | Crosslinked CC | 40 | 14 | 0.3 | 5.7 ± 0.6 |
| | 23 mg/mL | | | | |
| M2-B | Crosslinked CC/HA | 127 | 21 | 0.2 | 7.3 ± 0.6 |
| | 23 mg/mL | | | | |
| B9 | Crosslinked HA/free HA | 87 | 27 | 0.3 | 6.9 ± 1.0 |
| | 8 mg/mL | | | | |
| B10 | Crosslinked HA | 542 | 127 | 0.3 | 44 ± 20* |
| | 20 mg/mL | | | | |

*standard deviation is high, which is indicative of high friction between the two surfaces (low lubricating ability of the product tested);
**total concentration of polymers

It is observed that both crosslinked CC and co-crosslinked CC/HA hydrogels have a modulus of elasticity G′ in the same range as that of B9, while B10 has a higher modulus of elasticity. It is observed that both CC and CC/HA hydrogels exhibit significant lubricating ability, characterized by a low coefficient of friction between the two surfaces, comparable to that of the crosslinked HA viscosupplement B10, and better than that of the crosslinked HA viscosupplement B11.

In Examples 11 to 14, the polymers CC and HA used are those described in Tables 11a and 11b.

TABLE 11a
Table 11a - CC from Agaricus bisporus

| | DA | DA | DS | Intrinsic viscosity |
|---|---|---|---|---|
| Reference | level | (mol %) | (mol %) | (mL/g) |
| CC8 | DA < 40% | 28% ^{a} | 83% ^{b} | 900 to 1100 mL/g |
| CC1 | DA > 40% | 58% ^{c} | 82% ^{c} | |
| CC9 | | 55% ^{c} | 85% ^{c} | |
| CC5 | | 57% ^{c} | 88% ^{c} | |

^{a} value estimated from DA of starting chitosan:
^{b} value estimated from DS of CC after acetylation, measured by carbon-13 NMR;
^{c} measured by solid-phase carbon-13 NMR (formula 2).

TABLE 11b
Table 11b - Sodium hyaluronate (HA)

| | | Mw | Intrinsic viscosity |
|---|---|---|---|
| Type | Manufacturer | (million)* | (m³/kg)* |
| HA1 | HTL Javenech | −2.0 to 2.5 | 2.2 to 2.4 m³/kg |
| HA2 | (France) | −3.2 to 3.8 | 3.0 to 3.6 m³/kg |

### Example 11—Test of Co-Crosslinking HA with a CC Having a Degree of Acetylation of Less than 40%

It has been sought to verify whether it was possible to obtain a cohesive hydrogel by co-crosslinking CC and HA starting with a CC having DA less than 40% (CC8, Table 11a) and HA of HA1 type (Table 11 b), using the same conditions as those in Table 3a of Example 3. The conditions and characteristics of the obtained formulation are reported in Table 11c (reference M2-I), and compared with those of the reference hydrogel M2-A of Example 3 (in accordance with the invention).

It is observed that with CC8, a gel is not obtained by co-crosslinking and autoclave sterilization, as determined via the delta tangent value (tan delta, measured by rheometry. Indeed, the M2-I formulation includes a tan delta value of 1.6, that is higher than 1, indicative of a behavior of a viscous solution and not of a gel. On the contrary, the hydrogel M2-A has a tan delta value of 0.4, that is less than 1, indicative of a gel behavior, in accordance with the invention.

TABLE 11c
Table 11c Co-crosslinking of HA with a CC of DA < 40% (M2-I),
and comparison with the M2-A matrix of Example 3

| Reference | M2-1 | M2-A (Example 3) |
|---|---|---|
| CC (reference) | CC8 | CC1 |
| DA (mol (%) | DA < 40% | DA > 40% |
| HA (type) | HA1 | HA1 |
| Initial concentration of | 11% | 11% |
| polymers (%, m/v) | | |
| BDDE/polymers | 13% | 13% |
| (% g/100 g) | | |
| Sterilization by autoclave | A2 | A2 |
| Final concentration of | 24 | 23 |
| polymers (mg/mL) | | |
| Tan delta by rheometry:gel | Tan delta = 1.6 | Tan delta = 0.4 |
| (<1) or solution (>1) | (viscous, sticky | (gel) |
| | solution) | |
| Modulus of | 0.2 | 37 |
| elasticity G′ (Pa) | | |
| Viscoelasticity level | 0 | 2 |
| (scale from 0 to 4) | | |
| Hydrogel cohesion | Not measured* | OK |
| (water test) | | |

*the water test is not applicable because the formulation obtained is not a gel

### Example 12—Hydrogel for Volume Restoration or Filling of Large Skin Depressions

This example illustrates the use of a crosslinked CC-based hydrogel for restoring facial volumes or filling large skin depressions via subcutaneous injection or into the deep layers of the dermis. For these two indications, a level 4 viscoelasticity hydrogel is sought, that is with a modulus of elasticity G′ above about 150 Pa, while being cohesive according to the water test and easy to inject via a needle of 27 Gauge diameter and 13 mm length. In these indications, two commercial products B11 and B12 (Table 12), which are cohesive hydrogels based on crosslinked hyaluronan of elasticity level 4, are taken as references.

The hydrogel M2-J is obtained by co-crosslinking CC5 and HA type HA1 (CC/HA ratio 25:75) with 13% BDDE, at room temperature overnight. It has a modulus of elasticity of 295 Pa corresponding to the desired elasticity level 4, while remaining cohesive and easy to inject, in agreement with expectations for the intended indications (Table 12).

TABLE 12
Table 12 - Hydrogels for filling large skin
depressions or subcutaneous remodeling

| Reference | M2-J | B11 | B12 |
|---|---|---|---|
| CC (reference) | CC5 | / | / |
| Type of HA | HA1 | / | / |
| CC/HA ratio (m/m) | 25:75 | 0:100 | 0:100 |
| Initial concentration of | 11% | / | / |
| polymers (%, m/v) | | | |
| BDDE/polymers | 13% | / | / |
| (% g/100 g) | | | |
| Sterilization by autoclave | A2 | / | / |
| Final concentration of | 23 | 26 | 24 |
| polymer(s) (mg/mL) | | | |
| Hydrogel cohesion (water | OK | OK | OK |
| test) | | | |
| Easy to inject (27G needle, | OK | OK | OK |
| 13 mm) | | | |
| Tan delta | <1 | <1 | <1 |
| Modulus of elasticity G′ (Pa) | 295 | 300 | 190 |
| Viscoelasticity level | 4 | 4 | 4 |
| (scale from 0 to 4) | | | |

### Example 13—Volume Maintenance after Intradermal Injection of a Co-Crosslinked CC/HA Hydrogel Over a Period of 1 Month

A hydrogel is prepared by co-crosslinking CC9 (see Table 11a) and an HA2, with a CC/HA mass ratio of 40:60, according to the reaction conditions of Example 12. The hydrogel (reference M2-K) obtained is packaged in a 1 mL glass syringe (Hypak, BD Medical) and sterilized in the same manner as in Example 9. Its final concentration of polymer is 23 mg/mL, it is cohesive, injectable via a 27 G needle and has a viscoelasticity level of 3.

According to a protocol similar to that of Example 9, the same volume of hydrogel M2-K and commercial product B12 (see Table 12, viscoelasticity level of 4) are injected intradermally in rabbits, via a 27 Gauge needle. At regular intervals and for a period of 26 days after injection, the local reaction is evaluated, and then the volume of the papule formed by the injected product and visible on the surface of the skin is estimated by assigning it a score on a scale from 0 to 4. The volume of the papule is indicative of the presence of the product as well as its capacity to locally increase the volume of the skin tissue.

The injection of both products does not induce any significant local reaction during the follow-up period. Immediately after injection, a papule is formed with an average volume score of 3±0 for both products (out of 20 injection sites evaluated). In the days that followed, the papule resolves slightly, but actually remains present. At 26 days post-injection, the papule is still present, with a volume of mean score equal to 2.0±0.0 for M2-L and 2.4±0.5 for B12 (20 sites evaluated), which is consistent with their relative elasticity levels. The difference between the volume scores provided by hydrogels M2-K and B12 is not significant at this time point.

Thus, it is established that hydrogel M2-K actually remains present in the dermis and maintains a significant volumizing effect around its injection site for a duration of at least 26 days after intradermal injection in rabbits, as expected for an indication of filling skin depressions.

### Example 14—Preservation of a Co-Crosslinked CC/HA Hydrogel

The feasibility of preserving a co-crosslinked CC/HA hydrogel is evaluated by placing it in an accelerated aging condition in an oven at 40° C. and by monitoring the course of its biomechanical properties. The hydrogel is considered acceptable from a biomechanical point of view as long as it remains cohesive according to the water test and easily injectable, it includes a gel-like behavior (tan delta value lower than 1) and its viscoelasticity level is maintained with respect to the initial level at t0 and in agreement with the intended indication.

With the objective of obtaining a viscoelasticity level of 2, the reference hydrogel M2-L is prepared by co-crosslinking CC9 (see Table 11a) and a HA2 at a CC/HA ratio of 70:30, according to the reaction conditions of Example 12. This is a product packaged in a 1 mL glass syringe (Hypak, BD Medical) and sterilized, in the same manner as Example 9. The syringes are placed in an oven at 40° C. for a period of 6 months. The characteristics measured at the 3-month storage time are given in Table 13.

TABLE 13
Table 13 - Characteristics of a co-crosslinked CC/HA
hydrogel (M2-L) stored at 40° C. for 3 months

| Storage time at 40° C. | t0 | 1 month | 2 months | 3 months |
|---|---|---|---|---|
| Hydrogel cohesion | OK | OK | OK | OK* |
| (water test) | | | | |
| Easy to inject | OK | OK | OK | OK* |
| (27G needle, 13 mm) | | | | |
| Tan delta < 1 | OK | OK | OK | OK |
| Viscoelasticity level | 2 | 2 | 2 | 2 |
| (scale from 0 to 4) | | | | |

After 3 months under accelerated aging conditions at 40° C., the product M2-L remains a hydrogel (because tan delta<1) and its cohesion, ease of injection and viscoelasticity level of 2 are maintained. Therefore, it is estimated by extrapolation that this co-crosslinked CC/HA hydrogel should maintain acceptable properties for the intended indication for at least 12 months at room temperature.

1. A matrix comprising at least one carboxyalkyl chitosan having glucosamine units, N-acetyl glucosamine units and carboxyalkyl group substituted glucosamine units, said carboxyalkyl chitosan having a degree of acetylation greater than 40% and up to 80%, expressed as the number of moles of N-acetyl groups relative to the number of moles of total glucosamine units, said carboxyalkyl chitosan being crosslinked by covalent bonds between the carboxyalkyl chitosan chains.

2. The matrix according to claim 1, wherein said carboxyalkyl chitosan has a degree of substitution with a carboxyalkyl group greater than 20%, expressed as the number of moles of the substituent relative to the number of moles of total units.

3. The matrix according to claim 1, wherein the chitosan is derived from the mycelium of an Ascomycete fungus.

4. The matrix according to claim 1, wherein the carboxyalkyl chitosan is reacetylated.

5. The matrix according to claim 1, wherein the matrix is sterile.

6. The matrix according to claim 1, wherein the matrix forms a cohesive hydrogel.

7. The matrix according to claim 1, wherein the matrix comprises at least one hyaluronan.

8. The matrix according to claim 1, wherein the matrix comprises at least one hyaluronan obtained by fermentation.

9. The matrix according to claim 1, wherein the matrix comprises at least one hyaluronan crosslinked by covalent bonds.

10. The matrix according to claim 1, wherein the matrix comprises at least one hyaluronan co-crosslinked by covalent bonds with carboxyalkyl chitosan.

11. The matrix according to claim 1, wherein the crosslinks are formed by a crosslinking agent forming said covalent bonds.

12. The matrix according to claim 11, wherein the crosslinking agent is selected from the group consisting of crosslinking agents used for crosslinking polysaccharides, 1,4 butanediol diglycidyl ether, 1-bromo-3,4-epoxybutane, 1-bromo-4,5-epoxypentane, 1-chloro-2,3-epithio-propane, 1-bromo-2,3-epithiopropane, 1-bromo-3,4-epithio-butane, 1-bromo-4,5-epithiopentane, 2,3-dibromopropanol, 2,4-dibromobutanol, 2,5-dibromopentanol, 2,3-dibromopropanethiol, 2,4-dibromobutanethiol, 2,5-dibromopentane-thiol epichlorohydrin, 2,3-dibromopropanol, 1-chloro-2,3-epithiopropane, dimethylaminopropylcarbodiimide, gallic acid, epigallocatechin gallate, curcumin, tannic acid, genipin, diisocyanate compounds, hexamethylene diisocyanate, toluene diisocyanate, and even divinyl sulfone.

13. The matrix according to claim 10, wherein said matrix is capable of forming a hydrogel.

14. The matrix according to claim 10, wherein said matrix is capable of forming a cohesive hydrogel.

15. The matrix according to claim 1, wherein the matrix has an antioxidant capacity by scavenging free radicals.

16. A composition wherein it comprises at least one matrix defined according to claim 1.

17. The composition of claim 16 which is an injectable composition.

18. The composition of claim 16 which is a pharmaceutical composition.

19. The composition of claim 16 which is an injectable, implantable or instillable, or topically administrable pharmaceutical composition, or an injectable or implantable or instillable, or topically administrable medical device.

20. A method for the treatment, repair or filling of at least one body tissue in need of repair or filling, said method comprising injecting, implanting or instilling, or topically administering an effective amount of a pharmaceutical composition or medical device of claim 19.

21. The method of claim 20, wherein said method is for treating osteoarthritis, or repairing a cartilage defect.

22. A medical device, wherein it comprises a composition as defined in claim 16.

23. A process for preparing a matrix as defined in claim 1, said process comprising:
contacting the carboxyalkyl chitosan with at least one crosslinking agent;
crosslinking the carboxyalkyl chitosan with the crosslinking agent; and
obtaining a matrix comprising the carboxyalkyl chitosan crosslinked.

24. A process for preparing a matrix comprising a carboxyalkyl chitosan, preferably as defined in claim 1, co-crosslinked with another biopolymer, said process comprising:
contacting a mixture of carboxyalkyl and the other biopolymer with at least one crosslinking agent;
crosslinking the carboxyalkyl chitosan and the other biopolymer with the crosslinking agent; and
obtaining a co-crosslinked matrix of carboxyalkyl chitosan and the other biopolymer.
